(19) United States
(12) Patent Application Publication
(10) Pub. No.: US 2024/0138002 A1
(43) Pub. Date: Apr. 25, 2024

(54) PERSONAL DEVICE, A SYSTEM AND METHODS FOR HANDOVER BETWEEN A SELF-ORGANIZING NETWORK AND A MOBILE COMMUNICATIONS NETWORK

(71) Applicant: Pink Nectarine Health AB, Stockholm (SE)

(72) Inventors: Martin LINREY, Sollentuna (SE); Tobias LINGHAMMAR, Enebyberg (SE)

(73) Assignee: Pink Nectarine Health AB, Stockholm (SE)

(21) Appl. No.: 18/277,165

(22) PCT Filed: Feb. 14, 2022
(86) PCT No.: PCT/SE2022/050163

§ 371 Date: Aug. 13, 2023

(30) Foreign Application Priority Data
Feb. 15, 2021 (SE) 2150165-5

(51) Int. Cl. H04W 76/15; H04W 24/02; H04W 24/04; H04W 36/00; H04W 76/14; H04W 76/38

(52) U.S. Cl. CPC H04W 76/15; H04W 24/02; H04W 24/04; H04W 36/03; H04W 76/14; H04W 76/38

(57) ABSTRACT

Methods, a system, a self-organizing network of nodes and at least one portable personal device is provided. The at least one personal device is adapted to be worn by individuals and is configured to transmit short-range wireless broadcast signals. The personal device is configured to establish a short-range wireless communication link with the nodes of the self-organizing network and to enable the establishment of a short-range wireless communication link with a mobile communications device which may be associated with the personal device. The network and the personal devices are further configured to enable handover of the personal device between a node of the self-organizing network and a mobile communications device which may be associated with the personal device.

### TECHNICAL FIELD

The present invention relates to methods, a system, a self-organizing network of nodes and a portable personal device. Specifically, the present invention relates to a personal device, a system and methods for transmitting short-range wireless broadcast signals for controlling handover between a self-organizing network and a mobile communications network.

### BACKGROUND

Systems for monitoring such individuals, e.g. elderly, sick or injured people, and automatically issuing alarms have been used for some time. Monitoring systems for monitoring such individuals often comprise a personal device (e.g. a wristband) comprising sensors, the signals of which are analysed to determine whether help is needed, e.g. due to a fall.

U.S. Pat. No. 6,433,690 discloses a method and system for recording acceleration and body position data from elderly or disabled persons. The fall monitoring system includes signal feature extraction and interpretive methods for characterizing accelerations and body positions during fall events. The system can detect health and life-threatening fall events in elderly persons and can autonomously notify nursing personnel or family members that the person is in need of immediate assistance. The monitoring of a person's fall is performed by using an accelerometer in a monitoring device carried on the person, which monitoring device samples the person's body angle and body acceleration.

U.S. Pat. No. 9,668,297 describes a BLE scatternet system and method that uses a schedule for when advertisements should be transmitted. The advertiser transmits an initial advertisement, receives a connection request, and then goes to sleep to wake up and transmit subsequent advertisements according to the schedule.

U.S. Ser. No. 10/313,866 describes a method for establishing and monitoring a Bluetooth low energy wireless network by using the broadcast state.

US2019/0215244 describes a Self-Organizing Network (SON) for Internet of Things (IoT), where an IoT device may be selected to be an “IoT coordinator”. A coordinator may e.g. perform the roles of managing a neighboring IoT device and forwarding a packet. This SON method includes receiving neighboring device information from at least one neighboring IoT device of an IoT device, updating device information of the IoT device based on the received neighboring device information, and determining whether the IoT device is a coordinator candidate based on at least one of a number of network interfaces, a number of neighbor links connected to the network interfaces, or a ratio of remaining energy, which is included in the device information.

The managing of the IoT devices in US2019/0215244 is performed by neighboring IoT devices and, thus, relies on the presence of other IoT devices within short-range communication range and is also sharing short-range wireless transmission medium and protocol with other IoT devices thus is dependent on the currently available short-range bandwidth of those neighboring IoT devices, e.g. the current number of other links connected to the network interface of those neighboring IoT devices. US2016128043 describes the assignment of a dynamic ad hoc IoT gateway for providing inter-network communication between different IoT subnetworks. A dynamic ad hoc gateway aggregates communication within a proximal cloud associated with an IoT subnetwork to improve computational efficiency and support handoffs to another gateway node in response to topology changes, e.g., when one or more IoT devices leave and/or join the proximal cloud that defines the IoT subnetwork. The context associated with the IoT subnetwork may change from communicating with a trusted home network to an untrusted public network, from an untrusted public network to a trusted public network, etcetera. Handoff to a new gateway node may be facilitated for an IoT device in an IoT subnetwork prior to leaving the IoT subnetwork. The IoT device may then be configured to trigger a voting procedure to elect the new gateway node in response to the assigned gateway node leaving the IoT subnetwork.

There is a need for a personal device, system and methods for automatically performing personal device connection handover between a self-organizing network and a mobile communications device connected to a cellular mobile communications network.

More specifically, there is a need for a more scalable and robust system comprising a local self-organizing network which does not rely on the presence of inter-network gateways or other IoT devices within short-range communication range for making decisions on behalf of the IoT devices such as decisions related to automatically performing seamless handovers between a connection node of the local self-organizing network and a mobile communications device connected to a cellular mobile communications network.

### Problems with the Prior Art

It is difficult to design a personal device and system for performing handovers of the personal device carried by an individual moving between a local self-organizing network of nodes for monitoring the personal device in different locations or rooms of a facility and a mobile communications device connected to a cellular mobile communications network when the personal device is no longer within communication range of the local self-organizing network, e.g. when the personal device is outside the facility.

There is thus a need for a personal device, a system and methods for automatically performing handovers between a node of a local self-organizing network and a mobile communications device connected to a cellular mobile communications network. Specifically, there is a need for automatically performing seamless handover from a local self-organizing network to a mobile communications device connected to a cellular-type mobile communications network with reduced risk of losing connection to the cellular-type mobile communications network and a backend system communicatively coupled to the self-organizing network of nodes.

### SUMMARY

The above-described problem is addressed by the claimed system for monitoring individuals within predetermined facilities by transmitting data from personal devices worn by the individuals both via established short-range wireless connection links with the node network and via short-range wireless broadcasts.

The technology disclosed relates to methods for performing handover of a personal device, for example a wearable device such as a wristband, between a self-organizing network of nodes and a mobile communications device, and vice versa.

The technology disclosed relates to a system comprising a self-organizing network including at least two network nodes and a personal device which are configured to perform handover of the personal device between the self-organizing network and a mobile communications device by transmitting data from the personal device over an established short-range wireless connection links with a node of the node network and via the transmission of short-range wireless broadcasts from the personal device which are received by the nodes of self-organizing network within short-range communication range.

The technology disclosed relates to methods, a system comprising a self-organizing network of nodes and a plurality of personal devices each assigned one controller node and one connection node and configured to transmit data identifying and/or associated with the respective personal device both via established short-range wireless connection links with their respective assigned connection node and via short-range wireless broadcasts.

The technology disclosed also relates to a system and methods for transmitting data associated with a personal device worn by an individual both via an established short-range wireless connection link between the personal device and a network node and handling handovers of the personal device via the transmissions of short-range wireless signals from the personal device.

In aspects, the personal device is adapted to be worn by an individual and is configured to transmit short-range wireless signals which are used to control the handover of the personal device between a node of a local self-organizing network and a mobile communications device connected to a cellular mobile communications network.

In aspects, the present invention relates to methods, a system, a self-organizing network of nodes and a plurality of portable personal devices adapted to be worn by individuals and configured to transmit short-range wireless broadcast signals.

In embodiments, the technology disclosed relates to a personal device which is configured to first establish a short-range wireless communication link with a node of the self-organizing network and then enable the establishment of a short-range wireless communication link with a mobile communications device.

In embodiments, the personal devices are each configured to establish a short-range wireless communication link with the nodes of the self-organizing network and further configured to enable the establishment of a short-range wireless communication link with a mobile communications device associated with the respective personal device.

In embodiments, the self-organizing network and the personal devices are further configured to enable a seamless handover procedure between the nodes of the self-organizing network and a mobile communications device associated with the respective personal device.

In embodiments, the technology disclosed relates to a method in a system including a network comprising at least two network nodes, at least one personal device and at least one mobile communications device connected and/or coupled to a cellular-type mobile communications network, the method comprising:
a. establishing a short-range wireless communication link between the personal device and a network node of the network;
b. enabling, by the personal device, the establishing of a short-range wireless communication link between the personal device and the at least one mobile communications device;
c. transmitting, from the personal device, at least one short-range wireless broadcast signal containing a flag indicating that the personal device allows the establishing of a short-range wireless communication link with the at least one mobile communications device; and
d. establishing a short-range wireless communication link between the personal device and one of the at least one mobile communications device.

In embodiments, the technology disclosed relates to a method in a system including a network comprising at least two network nodes and at least one personal device linked to and/or associated with at least one mobile communications device connected and/or coupled to a cellular-type mobile communications network, the method comprising:
a. establishing a short-range wireless communication link between the personal device and a network node of the network;
b. enabling, by the personal device, the establishing of a short-range wireless communication link between the personal device and the at least one mobile communications device linked to and/or associated with the personal device;
c. transmitting, from the personal device, at least one short-range wireless broadcast signal containing a flag indicating that the personal device allows the establishing of a short-range wireless communication link with the at least one mobile communications device linked to and/or associated with the personal device; and
d. establishing a short-range wireless communication link between the personal device and one of the at least one mobile communications device communicatively linked to and/or associated with the personal device.

In embodiments, the technology disclosed relates to a method in a system including a network comprising at least two network nodes and at least one personal device with short-range wireless communication capabilities, the method comprising:
a. establishing, by a connection node assigned to the personal device, a short-range wireless communication link with the personal device;
b. enabling, by the personal device, the establishing of a short-range wireless communication link between the personal device and a mobile communications device; and
c. establishing a short-range wireless communication link between the personal device and a mobile communications device communicatively coupled to a cellular-type mobile communications network.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device;
c. initiating, by the wearable device, the enabling of short-range wireless communication between the wearable device and one of the at least one mobile communications device associated with the wearable device; and
d. establishing a short-range wireless communication link between the wearable device and a mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device during the transmission of data to the network node;
c. initiating, by the wearable device, the enabling of short-range wireless communication between the wearable device and one of the at least one mobile communications device associated with the wearable device, wherein said initiating of the enabling of short-range wireless communication does not include other mobile communications devices which are not associated with the wearable device; and
d. establishing a short-range wireless communication link between the wearable device and a mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently allow for communication with any mobile communications device;
c. initiating, by the wearable device, the enabling of short-range wireless communication between the wearable device and one of the at least one mobile communications device associated with the wearable device, wherein said initiating of the enabling of short-range wireless communication does not include other mobile communications devices which are not associated with the wearable device; and
d. establishing a short-range wireless communication link between the wearable device and a mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. communicating data over the established short-range wireless communication link, wherein the wearable device does not allow for communication with any mobile communications device as long as link conditions for the established link are not poor and/or a timer of the wearable device is not indicating that no messages were received by the wearable device for a certain time period;
c. initiating, by the wearable device, the enabling of short-range wireless communication between the wearable device and one of the at least one mobile communications device associated with the wearable device, wherein said initiating of the enabling of short-range wireless communication does not include other mobile communications devices which are not associated with the wearable device; and
d. establishing a short-range wireless communication link between the wearable device and a mobile communications device associated with the wearable device, wherein said establishment is following said step of initiating the enabling of short-range wireless communication between the wearable device and one of the at least one mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. communicating data over the established short-range wireless communication link, wherein the wearable device does not allow for communication with any mobile communications device as long as a timer of the wearable device is not indicating that no messages were received by the wearable device for a certain time window;
c. initiating, by the wearable device, the enabling of short-range wireless communication between the wearable device and one of the at least one mobile communications device associated with the wearable device, wherein said enabling is in response to the timer of the wearable device is indicating no messages were received by the wearable device for a certain time window, and wherein said initiating of the enabling of short-range wireless communication does not include the enabling of communication with any other mobile communications devices which are not associated with the wearable device; and
d. establishing a short-range wireless communication link between the wearable device and a mobile communications device associated with the wearable device, wherein said establishment is following said step of initiating the enabling of short-range wireless communication between the wearable device and one of the at least one mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. communicating data over the established short-range wireless communication link, wherein the wearable device does not allow for communication with any mobile communications device as long as a poor link conditions for the established short-range wireless communication link is not detected by the network of nodes;
c. initiating, by the wearable device, the enabling of short-range wireless communication between the wearable device and one of the at least one mobile communications device associated with the wearable device, wherein said enabling is in response to poor link conditions for the established short-range wireless communication link is not detected by the network of nodes, and wherein said initiating of the enabling of short-range wireless communication does not include the enabling of communication with any other mobile communications devices which are not associated with the wearable device; and
d. establishing a short-range wireless communication link between the wearable device and a mobile communications device associated with the wearable device, wherein said establishment is following said step of initiating the enabling of short-range wireless communication between the wearable device and one of the at least one mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. communicating data over the established short-range wireless communication link, wherein the wearable device does not allow for communication with any mobile communications device as long as a poor link conditions for the established short-range wireless communication link is not detected by the network node communicating with the wearable device over the established short-range wireless communication link;
c. initiating, by the wearable device, the enabling of short-range wireless communication between the wearable device and one of the at least one mobile communications device associated with the wearable device, wherein said enabling is triggered by or in response to poor link conditions for the established short-range wireless communication link is determined by the network node having the established short-range wireless communication link with the wearable device, and wherein said initiating of the enabling of short-range wireless communication does not include the enabling of communication with any other mobile communications devices which are not associated with the wearable device; and
d. establishing a short-range wireless communication link between the wearable device and a mobile communications device associated with the wearable device, wherein said establishment is following said step of initiating the enabling of short-range wireless communication between the wearable device and one of the at least one mobile communications device associated with the wearable device.

In embodiments, the technology disclosed relates to a method in a system including a network comprising at least two network nodes and a wearable device having an assigned controller node and which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. communicating data over the established short-range wireless communication link, wherein the wearable device does not allow for communication with any mobile communications device as long as a poor link conditions for the established short-range wireless communication link is not detected by the controller node assigned to the wearable device;
c. initiating, by the wearable device, the enabling of short-range wireless communication between the wearable device and one of the at least one mobile communications device associated with the wearable device, wherein said enabling is in response to poor link conditions for the established short-range wireless communication link is determined by the controller node assigned to the wearable device, and wherein said initiating of the enabling of short-range wireless communication does not include the enabling of communication with any other mobile communications devices which are not associated with the wearable device; and
d. establishing a short-range wireless communication link between the wearable device and a mobile communications device associated with the wearable device, wherein said establishment is following said step of initiating the enabling of short-range wireless communication between the wearable device and one of the at least one mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device;
c. initiating, by the wearable device, the enabling of short-range wireless communication between the wearable device and one of the at least one mobile communications device associated with the wearable device, wherein said enabling by the wearable device is triggered by and is in response to the expiry of a timer indicating that no messages have been received over the established short-range wireless communication link for a certain time period; and
d. establishing a short-range wireless communication link between the wearable device and a mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device;
c. initiating, by the wearable device, the enabling of short-range wireless communication between the wearable device and one of the at least one mobile communications device associated with the wearable device, wherein said enabling by the wearable device is triggered by and is in response to the determination of poor link conditions for the short-range wireless communication link; and
d. establishing a short-range wireless communication link between the wearable device and a mobile communications device associated with the wearable device.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device;
c. initiating, by the wearable device, the enabling of short-range wireless communication between the wearable device and a mobile communications device associated with the wearable device, wherein said enabling by the wearable device is triggered by and is in response to the expiry of a timer indicating that no messages have been received over the established short-range wireless communication link for a certain time period;
d. transmitting, from the wearable device, at least one short-range wireless broadcast signal containing a bit or flag indicating to a mobile communications device associated with the wearable device that the wearable device allows for the establishment of a short-range wireless communication link with the mobile communications device;
e. receiving, by a mobile communications device associated with the wearable device, a short-range wireless broadcast signal containing a bit or flag indicating to the mobile communications device that the wearable device allows for the establishment of a short-range wireless communication link with the mobile communications device; and
f. establishing a short-range wireless communication link between the wearable device and a mobile communications device associated with the wearable device, wherein the establishment of a short-range wireless communication with the mobile communications device associated with the wearable device is triggered by the associated mobile communications device receiving the short-range wireless broadcast signal containing the bit or flag.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device;
c. initiating, by the wearable device, the enabling of short-range wireless communication between the wearable device and a mobile communications device associated with the wearable device, wherein said enabling by the wearable device is triggered by and is in response to the determination of poor link conditions for the short-range wireless communication link;
d. transmitting, from the wearable device, at least one short-range wireless broadcast signal containing a bit or flag indicating to a mobile communications device associated with the wearable device that the wearable device allows for the establishment of a short-range wireless communication link with the mobile communications device;
e. receiving, by a mobile communications device associated with the wearable device, a short-range wireless broadcast signal containing a bit or flag indicating to the mobile communications device that the wearable device allows for the establishment of a short-range wireless communication link with the mobile communications device; and
f. establishing a short-range wireless communication link between the wearable device and a mobile communications device associated with the wearable device, wherein the establishment of a short-range wireless communication with the mobile communications device associated with the wearable device is triggered by the associated mobile communications device receiving the short-range wireless broadcast signal containing the bit or flag.

In embodiments, the technology disclosed relates to a method in a system including a network comprising at least two network nodes and a wearable device which is linked to or associated with at least one mobile communications device having cellular communications capabilities configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device;
c. initiating, by the wearable device, the enabling of short-range wireless communication between the wearable device and a mobile communications device associated with the wearable device, wherein said enabling by the wearable device is triggered by and is in response to at least one of the expiry of a timer indicating that no messages have been received over the established short-range wireless communication link for a certain time period and the determination of poor link conditions for the short-range wireless communication link;
d. transmitting, from the wearable device, at least one short-range wireless broadcast signal containing a bit or flag indicating to a mobile communications device associated with the wearable device that the wearable device allows for the establishment of a short-range wireless communication link with the mobile communications device;
e. receiving, by a mobile communications device associated with the wearable device, a short-range wireless broadcast signal containing a bit or flag indicating to the mobile communications device that the wearable device allows for the establishment of a short-range wireless communication link with the mobile communications device; and
f. establishing a short-range wireless communication link between the wearable device and a mobile communications device associated with the wearable device, wherein the establishment of a short-range wireless communication with the mobile communications device associated with the wearable device is triggered by the associated mobile communications device receiving the short-range wireless broadcast signal containing the bit or flag.

In embodiments, the technology disclosed relates to a method in a system including a network comprising at least two network nodes and a wearable device which is linked to or associated with at least one mobile communications device having cellular communications capabilities and configured to be connected to and/or communicatively coupled to a cellular-type mobile communications network, the method comprising:
a. establishing a short-range wireless communication link between the wearable device and a network node of the network;
b. transmitting, by the wearable device, data over the established short-range wireless communication link with the network node, wherein the wearable device does not currently enable communication with any mobile communications device;
c. initiating, by the wearable device, the enabling of short-range wireless communication between the wearable device and the at least one mobile communications device associated with the wearable device, wherein said enabling by the wearable device does not include other mobile communications devices that are not associated with the wearable device and is triggered by or is in response to at least one of the expiry of a timer of the wearable device is indicating that no messages have been received over the established short-range wireless communication link for a certain time period and the determination, by at least one of a network node connected to or assigned to the wearable device, of poor link conditions for the short-range wireless communication link;
d. transmitting, from the wearable device, at least one short-range wireless broadcast signal containing a bit or flag indicating to a mobile communications device associated with the wearable device that the wearable device allows for the establishment of a short-range wireless communication link with the mobile communications device;
e. receiving, by a mobile communications device associated with the wearable device, a short-range wireless broadcast signal containing a bit or flag indicating to the mobile communications device that the wearable device allows for the establishment of a short-range wireless communication link with the mobile communications device; and
f. establishing a short-range wireless communication link between the wearable device and a mobile communications device associated with the wearable device, wherein the establishment of a short-range wireless communication with the mobile communications device associated with the wearable device is triggered by the associated mobile communications device receiving the short-range wireless broadcast signal containing the bit or flag.

In embodiments, the technology disclosed relates to a method in a system including a network comprising at least two network nodes and at least one wearable device with short-range wireless communication capabilities and which is associated with at least one mobile communications device, the method comprising:
a. transmitting, from a wearable device, at least one short-range wireless broadcast signal;
b. receiving, by at least one node of the at least two network nodes, said at least one short-range wireless broadcast signal;
c. assigning one node among the at least two network nodes to be controller node for said wearable device;
d. assigning a connection node for establishing a short-range wireless communication link between said connection node and said wearable device;
e. establishing, by said assigned connection node, a short-range wireless communication link with said wearable device;
f. transmitting, from said wearable device and via said established short-range wireless communication link, a message to said connection node;
g. receiving, by said connection node, said message, wherein said message contains data including at least one of status data, sensor data and event data associated with said wearable device;
h. transmitting, from the assigned connection node to the controller node for said wearable device, data including said at least one of status data, sensor data and event data associated with said wearable device;
i. enabling, by said wearable device, the establishing of a short-range wireless communication link between said wearable device and a mobile communications device associated with the wearable device, wherein said enabling does not include mobile communications devices which are not associated with the wearable device, and wherein said step of enabling is in response to and triggered by at least one a timer duration period is exceeded without any messages over said established short-range wireless communication link is received and the determining of poor link conditions for said established short-range wireless communication link; and
j. establishing, following said step of enabling, a short-range wireless communication link between said wearable device and a mobile communications device associated with the wearable device and which is communicatively coupled to a cellular-type mobile communications network.

In embodiments, the technology disclosed relates to a wearable device associated with at least one mobile communications device and with short-range wireless communication capabilities for use in a system including a local network comprising at least two network nodes, wherein said wearable device is configured to:
a. transmit at least one short-range wireless broadcast signal;
b. establish a first short-range wireless communication link with a node of the network;
c. transmitting data over the first short-range wireless communication link without allowing for communication with any mobile communications device, said wearable device is further configured to:
d. enable the establishment of short-range wireless communication between said wearable device and a mobile communications device associated with the wearable device, wherein said enabling does not include communication with any other mobile communications devices which are not associated with the wearable device, and wherein said step of enabling is in response to and triggered by at least one a timer duration period is exceeded without any messages over said established short-range wireless communication link is received and the determining of poor link conditions for said established short-range wireless communication link;
e. transmit at least one short-range wireless broadcast signal containing a flag indicating that the wearable device allows for the establishing of a short-range wireless communication link with a mobile communications device associated with the wearable device and which is connected to a cellular mobile communications network; and
f. establish a short-range wireless communication link with a mobile communications device associated with the wearable device.

In embodiments, the technology disclosed relates to a system including a local network comprising at least two network nodes and a wearable device associated with at least one mobile communications device, said system is configured to assign a connection node among said at least two network nodes to each of the at least one wearable device, wherein the assigned connection node is responsible for establishing a short-range wireless communication link with their respective wearable device, and each of the at least one wearable device is configured to transmit short-range wireless broadcast signals while having an active short-range wireless connection with a connection node assigned to the wearable device, said system is further configured to:
a. establish a short-range wireless communication link between the wearable device and a connection node assigned to said wearable device;
b. obtain, by the connection node assigned to said wearable device, signal strength values and/or packet loss rates over said short-range wireless communication link;
c. determine, by the controller node assigned to said wearable device, that a link condition of said short-range wireless communication link is poor based on at least one of the signal strength values and/or high packet loss rates obtained by the connection node; and, in response to said determining that the link condition is poor:
d. transmit, from said controller node assigned to said wearable device via said established short-range wireless communication link between said connection node and said wearable device, a message containing data instructing said wearable device to allow for the establishing of a short-range wireless communication link with a mobile communications device connected to a cellular mobile communications network, wherein said message is only enabling short-range wireless communication link with a mobile communications device which is associated with said wearable device and not with any other mobile communications devices whish are not associated with the wearable device; and
e. establish a second simultaneous short-range wireless communication link between said wearable device and a mobile communications device associated with said wearable device and which is connected to a cellular mobile communications network.

In embodiments, the technology disclosed relates to a method in a system including a network comprising at least two network nodes and at least one personal device linked to and/or associated with at least one mobile communications device connected and/or coupled to a cellular-type mobile communications network, the method comprising:
a. establishing, by a connection node assigned to the personal device, a short-range wireless communication link with the personal device;
b. enabling, by the personal device, the establishing of a short-range wireless communication link between the personal device and the at least one mobile communications device linked to and/or associated with the personal device; and
c. establishing a short-range wireless communication link between the personal device and one of the at least one mobile communications device communicatively linked to and/or associated with the personal device.

In embodiments, the technology disclosed relates to a method in a system including a network comprising at least two network nodes and at least one personal device linked to and/or associated with at least one mobile communications device connected and/or coupled to a cellular-type mobile communications network, the method comprising:
a. establishing, by a connection node assigned to the personal device, a short-range wireless communication link with the personal device;
b. enabling, by the personal device, the establishing of a short-range wireless communication link between the personal device and the at least one mobile communications device linked to and/or associated with the personal device, wherein the personal device does not enabled the establishment of other mobile communications devices than the at least one mobile communications device linked to and/or associated with the personal device; and
c. establishing a short-range wireless communication link between the personal device and one of the at least one mobile communications device communicatively linked to and/or associated with the personal device.

In embodiments, the technology disclosed relates to a method in a system including a network comprising at least two network nodes and at least one personal device linked to and/or associated with at least one mobile communications device connected and/or coupled to a cellular-type mobile communications network, the method comprising:
a. assigning a node of the network to be a controller node for the personal device;
b. assigning, by the assigned controller node, a node of the network to be the connection node for the personal device, wherein the controller node and connection node may or may not be the same node;
c. establishing, by the connection node assigned to the personal device, a short-range wireless communication link with the personal device;
d. enabling, by the personal device, the establishing of a short-range wireless communication link between the personal device and the at least one mobile communications device linked to and/or associated with the personal device;
e. transmitting, from the personal device, at least one short-range wireless broadcast signal containing a flag indicating that the personal device allows the establishing of a short-range wireless communication link with the at least one mobile communications device linked to and/or associated with the personal device; and
f. establishing a short-range wireless communication link between the personal device and one of the at least one mobile communications device communicatively linked to and/or associated with the personal device.

In embodiments, the technology disclosed relates to a method in a system including a network comprising at least two network nodes and at least one personal device with short-range wireless communication capabilities, the method comprising:
a. establishing, by a connection node assigned to the personal device, a short-range wireless communication link with the personal device;
b. enabling, by the personal device, the establishing of a short-range wireless communication link between the personal device and a mobile communications device; and
c. establishing a short-range wireless communication link between the personal device and a mobile communications device communicatively coupled to a cellular-type mobile communications network.

In embodiments, the technology disclosed relates to a method in a system including a network comprising at least two network nodes and at least one personal device with short-range wireless communication capabilities, the method comprising:
a. assigning a node of the network to be a controller node for the personal device;
b. assigning a node of the network to be the connection node for the personal device, wherein the controller node and connection node may or may not be the same node;
c. establishing a short-range wireless communication link between the connection node and the personal device;
d. determining, by the personal device, that the established short-range wireless communication link with the connection node is disconnected; and
e. establishing a short-range wireless communication link between the personal device and a mobile communications device communicatively coupled to a cellular-type mobile communications network.

In embodiments, the technology disclosed relates to a method in a system including a network comprising at least two network nodes and at least one personal device with short-range wireless communication capabilities, the method comprising:
a. assigning a node of the network to be a controller node for the personal device;
b. assigning a node of the network to be the connection node for the personal device, wherein the controller node and connection node may or may not be the same node;
c. establishing, by a connection node of the network assigned to the personal device, a short-range wireless communication link with the personal device;
d. determining, by the controller node, that the link conditions of the established short-range wireless communication link with the connection node is poor; and
e. establishing a short-range wireless communication link between the personal device and a mobile communications device communicatively coupled to a cellular-type mobile communications network.

In embodiments, the technology disclosed relates to a method in a system including a network comprising at least two network nodes and at least one personal device with short-range wireless communication capabilities, the method comprising:
a. assigning a node of the network to be a controller node for the personal device;
b. assigning a node of the network to be the connection node for the personal device, wherein the controller node and connection node may or may not be the same node;
c. establishing, by a connection node of the network assigned to the personal device, a short-range wireless communication link with the personal device;
d. obtaining, by the connection node, at least one of low signal strength values and high packet loss rates over the short-range wireless communication link;
e. determining, by the controller node, that the link conditions of the established short-range wireless communication link with the connection node is poor; and
f. establishing a short-range wireless communication link between the personal device and a mobile communications device communicatively coupled to a cellular-type mobile communications network.

In embodiments, the technology disclosed relates to a method in a system including a network comprising at least two network nodes and at least one personal device with short-range wireless communication capabilities, the method comprising:
a. assigning a node of the network to be a controller node for the personal device;
b. assigning a node of the network to be the connection node for the personal device, wherein the controller node and connection node may or may not be the same node;
c. establishing, by a connection node of the network assigned to the personal device, a short-range wireless communication link with the personal device;
d. obtaining, by the connection node, at least one of signal strength values and high packet loss rates over the short-range wireless communication link;
e. transmitting, from the connection node to the controller node, data indicating the at least one of signal strength values and packet loss rates over the short-range wireless communication link;
f. determining, by the controller node and based on the received data indicating the at least one of signal strength values and packet loss rates, that the link conditions of the established short-range wireless communication link with the connection node is poor; and
g. establishing a short-range wireless communication link between the personal device and a mobile communications device communicatively coupled to a cellular-type mobile communications network.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal;
b. receiving, by at least one node of the at least two network nodes, the at least one short-range wireless broadcast signal;
c. assigning one node among the at least two network nodes to be controller node for the personal device;
d. assigning, by the assigned controller node, one node among the at least two network nodes to be a connection node for the personal device, wherein the assigned controller node is responsible for establishing a short-range wireless communication link between the connection node and the personal device;
e. establishing, by the assigned connection node, a short-range wireless communication link with the personal device;
f. transmitting, from the personal device and via the established short-range wireless communication link, a message to the connection node;
g. receiving, by the connection node, the message, wherein the message contains data including at least one of status data, sensor data and event data associated with the personal device;
h. transmitting, from the assigned connection node to the controller node for the personal device, data including the at least one of status data, sensor data and event data associated with the personal device;
i. enabling, by the personal device, the establishing of a short-range wireless communication link between the personal device and a mobile communications device; and
j. establishing a short-range wireless communication link between the personal device and a mobile communications device communicatively coupled to a cellular-type mobile communications network.

In embodiments, the technology disclosed relates to methods and a system comprising at least two network nodes and a personal device configured to transmit a short-range wireless broadcast signal containing a flag indicating that the personal device allows the establishing of a short-range wireless communication link with a mobile communications device.

In embodiments, the technology disclosed relates to a system, a personal device and method where the step of enabling, by the personal device, the establishing of a short-range wireless communication link with a mobile communications device is in response to and triggered by a timer duration period of a timer of the personal device is exceeded without the personal device is receiving any messages over the short-range wireless communication link.

In embodiments, the technology disclosed relates to a system, a personal device and method where the step of enabling, by the personal device, the establishing of a short-range wireless communication link with a mobile communications device is followed by the step of:
a. receiving, by the personal device and from a mobile communications device, a connection request in the form of a broadcast signal for establishing a short-range wireless communication link with the mobile communications device.

In embodiments, the technology disclosed relates to a system, a personal device and method where the step of enabling, by the personal device, the establishing of a short-range wireless communication link with a mobile communications device is in response to and triggered by a timer duration period of a timer of at least one of the connection node assigned to the personal device, the controller node assigned to the personal device and a locally-installed or remote processing arrangement communicatively coupled to the controller node is exceeded without the assigned connection node is receiving any messages over the short-range wireless communication link.

In embodiments, the technology disclosed relates to a system, a personal device and method where the step of enabling, by the personal device, of a short-range wireless communication link with a mobile communications device is in response to and triggered by:
b. receiving, by the personal device and via the established short-range wireless communication link with the connection node assigned to the personal device, a message containing data adapted to instruct the personal device to enable a short-range wireless communication link with a mobile communications device.

In embodiments, the technology disclosed relates to a system, a personal device and method where the above message and/or instruction data is transmitted from at least one of the controller node assigned to the personal device and a locally-installed or remote processing arrangement which is communicatively coupled to the controller node assigned to the personal device.

In embodiments, the technology disclosed relates to a system, a personal device and method where the step of establishing a short-range wireless communication link between the personal device and the mobile communications device is preceded by and in response to the following steps:
c. transmitting, from the personal device, at least one short-range wireless broadcast signal containing a flag indicating that the personal device allows the establishing of a short-range wireless communication link with a mobile communications device; and
d. receiving, by a mobile communications device connected to a cellular mobile communications network, the at least one short-range wireless broadcast signal containing the flag.

In embodiments, the technology disclosed relates to a system, a personal device and method where the mobile communications device transmits a connection request to the personal device in response to receiving the at least one short-range wireless broadcast signal containing the flag indicating that the personal device allows the establishing of a short-range wireless communication link with the mobile communications device.

In embodiments, the flag of the at least one short-range wireless broadcast signal is adapted to indicate only to a mobile communications device which is associated with, or known to, the personal device that the personal device allows the establishing of a short-range wireless communication link. The flag may then be adapted so that it does not indicate to other mobile communications devices which are not associated with the personal device that they can establish a short-range wireless communication link with the personal device.

In different embodiments, the flag may be bit values or flag field in a broadcast message, e.g. using the Bluetooth Low Energy (BLE), which indicates to a mobile communications device that the personal device is enabled to establish a connection with the mobile communications device, e.g. a mobile communications device associated with the personal device. In embodiments and in response to receiving the broadcast message with the flag, the mobile communication device transmits a Bluetooth connection request to the personal device for establishing a

In embodiments, the technology disclosed relates to a system, a personal device and method where step of transmitting at least one short-range wireless broadcast signal containing a flag is preceded by the step of:
e. determining, by the personal device, that the short-range wireless communication link is disconnected based on a preset timer duration period is exceeded without receiving any messages over the short-range wireless communication link.

In embodiments, the at least one short-range wireless broadcast signal containing a flag is transmitted in response to the step of determining that the short-range wireless communication link is disconnected based on a preset timer duration period is exceeded without receiving any messages over the short-range wireless communication link.

In embodiments, the technology disclosed relates to a system, a personal device and method comprising the following step after establishing of the short-range wireless communication link between the personal device and the mobile communications device:
f. enabling, by the personal device, a second simultaneous short-range wireless communication link.

In embodiments, the second short-range wireless communication link is established in response to and following the further step of:
g. determining, by the controller node, that the link conditions of the short-range wireless communication link is poor based on at least one of low signal strength values and high packet loss rates over the short-range wireless communication link obtained by the connection node.

In embodiments, the at least one short-range wireless broadcast signal containing a flag is transmitted in response to the step of determining that the link condition of the short-range wireless communication link is poor based on at least one of low signal strength values and high packet loss rates over the short-range wireless communication link obtained by the connection node.

In embodiments, the second short-range wireless communication link with the mobile communications device is established while the short-range wireless communication link with the connection node is still active and/or has not been actively disconnected.

In embodiments, the method is further comprising the following step preceding the step of transmitting at least one short-range wireless broadcast signal containing a flag:
h. transmitting, from the controller node via the connection node to the personal device, a message containing data instructing the personal device to enable the establishing of a simultaneous short-range wireless communication link with a mobile communications device.

In embodiments, the personal device has an established short-range wireless communication link with the mobile communications device and the method is further comprising the step of:
i. receiving, by one of the at least two network nodes of the network, at least one short-range wireless broadcast signal from the personal device.

In embodiments, the personal device has an established short-range wireless communication link with the mobile communications device and the method is further comprising:
j. determining, by the network, that the signal strength values of the at least one short-range wireless broadcast signal from the personal device is sufficiently good, e.g. above a pre-determined signal strength threshold value or below a pre-determined packet loss threshold value;
k. establishing a new short-range wireless communication link between the personal device and the network by assigning a controller node and a connection node to the personal device.

In embodiments, the personal device has an established short-range wireless communication link with the mobile communications device and the method is further comprising:
l. determining, by the controller node, that the link condition of the short-range wireless communication link between the personal device and the connection node is sufficiently good, e.g. above a pre-determined threshold value, again based on at least one of signal strength values and packet loss rates over the short-range wireless communication link obtained by the connection node; and
m. re-connecting the personal device to the network, wherein the re-connecting step is performed in response to and is preceded by the step of determining that the link condition is sufficiently good.

In certain embodiments, the flag of the short-range wireless broadcast signal is indicating that only a mobile communications device which is associated with, or known to, the personal device is allowed to establish a connection with the personal device and that other mobile communications devices which are not associated with the personal device are not allowed to establish a connection to the personal device.

In embodiments, the flag indicating that the personal device allows and/or is enabled for the establishing of a short-range wireless communication link with a mobile communications device may be a predefined flag field value in the short-range wireless broadcast signal/message.

In certain embodiments, the personal device may then be configured to set a predefined flag field value in the broadcast message/signal, e.g. a bit value, which indicates to the receiver of the short-range wireless broadcast message/signal that the personal device allows, the establishing of a short-range wireless communication link with a mobile communications device.

In certain embodiments, the flag field value may then be adapted to indicate to a mobile communications device associated with the personal device that the personal device allows for the establishing of a short-range wireless communication link with the associated mobile communications device, whereas the flag field value in the short-range wireless broadcast signal/message does not indicate to other mobile communications devices not associated with the personal device receiving the short-range wireless broadcast signal/message with the flag that the personal device allows for the establishing of a short-range wireless communication link with the personal device.

In embodiments, the handover to the mobile communications device is initiated and performed in response to the step of determining that an established short-range wireless communication link to a connection node of the self-organizing network is disconnected, or that the link condition is poor based on at least one of low signal strength values and high packet loss rates over the short-range wireless communication link.

In embodiments, the handover to the mobile communications device is initiated and performed in response to the step of determining that the link condition of an established short-range wireless communication link with a connection node of the self-organizing network is poor where the determining of the link condition is based on at least one of low signal strength values and high packet loss rates over the short-range wireless communication link.

In embodiments, the transmission of the at least one short-range wireless broadcast signal containing a flag is initiated and performed while the personal device has an established communication connection or link with a node of the self-organizing network. The transmission of broadcast signals containing a flag indicating that the personal device allows for the establishing of a short-range wireless communication link with a mobile communications device, e.g. that the personal device allows for the establishing of a short-range wireless communication link only with a mobile communications device which is associated with the personal device, may then be performed in parallel with or while the personal device is transmitting messages via the established connection or link.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal containing a flag indicating that the personal device allows the establishing of a short-range wireless communication link with a mobile communications device; and
b. establishing a short-range wireless communication link between the personal device and a mobile communications device.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal containing a flag indicating that the personal device allows the establishing of a short-range wireless communication link with a mobile communications device associated with the personal device; and
b. establishing a short-range wireless communication link between the personal device and the mobile communications device associated with the personal device.

In embodiments, the at least one short-range wireless broadcast signal containing a flag indicating that the personal device allows the establishing of a short-range wireless communication link with a mobile communications device is transmitted in response to and/or preceded by determining, by the personal device, that the short-range wireless communication link is disconnected based on a preset timer duration period is exceeded without receiving any messages over the short-range wireless communication link.

In embodiments, the at least one short-range wireless broadcast signal containing a flag indicating that the personal device allows the establishing of a short-range wireless communication link with a mobile communications device is transmitted in response to and/or preceded by determining, by a controller node assigned to the personal device, that the link condition of the short-range wireless communication link is poor based on at least one of low signal strength values and high packet loss rates over the short-range wireless communication link obtained by the connection node assigned to the personal device. The connection node and the controller node may then be different nodes in a local self-organizing network comprising at least two nodes, or may be the same node.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal;
b. receiving, by at least one node of the at least two network nodes, the at least one short-range wireless broadcast signal;
c. assigning one node among the at least two network nodes to be controller node for the personal device;
d. assigning a connection node for establishing a short-range wireless communication link between the connection node and the personal device;
e. establishing, by the assigned connection node, a short-range wireless communication link with the personal device;
f. transmitting, from the personal device and via the established short-range wireless communication link, a message to the connection node;
g. receiving, by the connection node, the message, wherein the message contains data including at least one of status data, sensor data and event data associated with the personal device;
h. transmitting, from the assigned connection node to the controller node for the personal device, data including the at least one of status data, sensor data and event data associated with the personal device;
i. enabling, by the personal device, the establishing of a short-range wireless communication link between the personal device and a mobile communications device; and
j. establishing a short-range wireless communication link between the personal device and a mobile communications device communicatively coupled to a cellular-type mobile communications network.

In embodiments, the above step of enabling, by the personal device, of a short-range wireless communication link with a mobile communications device is in response to and triggered by a timer duration period of a timer of the personal device is exceeded without the personal device receiving any messages over the short-range wireless communication link.

In embodiments, the controller node for the personal device is configured to initiate a handover of the personal device from the connection node to a mobile communications device by transmitting a message containing data adapted to instruct the personal device to enable a short-range wireless communication link with a mobile communications device in response to determining, by the controller node, that:
no signal strength values from the other nodes have been received for a certain timer period or that the received signal strength values from the other nodes measuring the broadcast signals from the personal device during a timer period are below a certain threshold value; and
that the signal strength values and/or packet loss rates for the established short-range wireless communication link with the connection node assigned to the personal device is indicating poor link conditions.

In embodiments, the above step of enabling, by the personal device, of a short-range wireless communication link with a mobile communications device is in response to and triggered by:
a. receiving, by the personal device and via the established short-range wireless communication link with the connection node, a message containing data adapted to instruct the personal device to enable a short-range wireless communication link with a mobile communications device, wherein the message and/or instruction data is transmitted from the controller node assigned to the personal device.

In embodiments, the above step of establishing a short-range wireless communication link between the personal device and the mobile communications device is preceded by and in response to the following steps:
a. transmitting, from the personal device, at least one short-range wireless broadcast signal containing a flag indicating that the personal device allows the establishing of a short-range wireless communication link with a mobile communications device; and
b. receiving, by a mobile communications device connected to a cellular mobile communications network, the at least one short-range wireless broadcast signal containing the flag.

In embodiments, the controller node for the personal device is configured to initiate a handover of the personal device from the connection node to a mobile communications device in response to determining, by the controller node, that:
no signal strength values from the other nodes have been received for a certain timer period or that the received signal strength values from the other nodes measuring the broadcast signals from the personal device during a timer period are below a certain threshold value; and
that the signal strength values and/or packet loss rates for the established short-range wireless communication link with the connection node assigned to the personal device is indicating poor link conditions.

In embodiments, the mobile communications device transmits a connection request to the personal device in response to receiving the at least one short-range wireless broadcast signal containing the flag indicating that the personal device allows the establishing of a short-range wireless communication link with the mobile communications device.

In embodiments, the flag of the at least one short-range wireless broadcast signal is adapted to indicate only to a mobile communications device which is associated with, or known to, the personal device that the personal device allows the establishing of a short-range wireless communication link. In certain embodiments, the flag does not indicate to other mobile communications devices which are not associated with the personal device that they can establish a short-range wireless communication link with the personal device.

In embodiments, the above step of transmitting at least one short-range wireless broadcast signal containing a flag is preceded by the step of:
a. determining, by the personal device, that the short-range wireless communication link is disconnected based on a preset timer duration period is exceeded without receiving any messages over the short-range wireless communication link.

In embodiments, the at least one short-range wireless broadcast signal containing a flag is transmitted in response to the above step of determining that the short-range wireless communication link is disconnected based on a preset timer duration period is exceeded without receiving any messages over the short-range wireless communication link.

In embodiments, the method comprises the following step after the establishing of the short-range wireless communication link between the personal device and the mobile communications device:
a. enabling, by the personal device, a second simultaneous short-range wireless communication link.

In embodiments, the above-mentioned second short-range wireless communication link is established in response to and following the further step of:
a. determining, by the controller node, that the link condition of the short-range wireless communication link is poor based on at least one of low signal strength values and high packet loss rates over the short-range wireless communication link obtained by the connection node.

In embodiments, the at least one short-range wireless broadcast signal which contains a flag is transmitted in response to the above step of determining that the link condition of the short-range wireless communication link is poor based on at least one of low signal strength values and high packet loss rates over the short-range wireless communication link which are obtained by the connection node.

In certain embodiments the at least one short-range wireless broadcast signal which contains a flag is transmitted in response to the above step of determining, by the controller node, that the link condition of the short-range wireless communication link is poor and there is no other network node which receives short-range wireless broadcast signal from the personal device having signal strength values which are above a certain threshold value and/or are stronger than the signal strength values for the broadcast signals measured by the connection node.

In embodiments, the second short-range wireless communication link with the mobile communications device is established while the short-range wireless communication link with the connection node is still active and/or has not been actively disconnected.

In embodiments, the method further comprises the following step preceding the step of transmitting at least one short-range wireless broadcast signal containing a flag:
b. transmitting, from the controller node via the connection node to the personal device, a message containing data instructing the personal device to enable the establishing of a simultaneous short-range wireless communication link with a mobile communications device.

In embodiments where the personal device has an established short-range wireless communication link with the mobile communications device, the method further comprises the step of:
c. receiving, by one of the at least two network nodes of the network, at least one short-range wireless broadcast signal from the personal device.

In embodiments, the method is further comprising:
d. determining, by the network, that the signal strength values of the at least one short-range wireless broadcast signal from the personal device is sufficiently good;
e. establishing a new short-range wireless communication link between the personal device and the network by assigning a controller node and a connection node to the personal device.

In embodiments, the method further comprises:
f. determining, by the controller node, that the link condition of the short-range wireless communication link between the personal device and the connection node is sufficiently good again based on at least one of signal strength values and packet loss rates over the short-range wireless communication link obtained by the connection node; and
g. re-connecting the personal device to the network, wherein the re-connecting step is performed in response to and is preceded by the step of determining that the link condition is sufficiently good.

In embodiments, the method further comprises disconnecting the short-range wireless communication link between the personal device and the mobile communications device.

In embodiments, the short-range wireless communication link is disconnected in response to and following the above establishing of a new short-range wireless communication link between the personal device and the network.

In aspects, the technology disclosed relates to a personal device, e.g. a wristband, with short-range wireless communication capabilities for use in a system including a local network comprising at least two network nodes, where the system is configured to assign a connection node among the at least two network nodes to the personal device, wherein the connection node is responsible for establishing a short-range wireless communication link with their respective personal device, and each of the plurality of personal devices is configured to transmit short-range wireless broadcast signals while their respective established short-range wireless connection with their respectively assigned connection node is active, wherein the personal device is configured to:
g. transmit at least one short-range wireless broadcast signal;
h. establish a first short-range wireless communication link with a connection node of the network, and wherein the personal device is further configured to:
i. transmit at least one short-range wireless broadcast signal containing a flag indicating that the personal device allows the establishing of a short-range wireless communication link with a mobile communications device communicatively coupled to a cellular-type mobile communications network.

In embodiments, the personal device further comprises a timer configured to:
a. determine that the short-range wireless communication link with a connection node of the network is disconnected based on a preset timer duration period of the timer is exceeded without receiving any messages over the short-range wireless communication link.

In embodiments, the personal device is further configured to transmit the at least one short-range wireless broadcast signal containing a flag in response to the above step of determining that the short-range wireless communication link is disconnected based on a preset timer duration period is exceeded without receiving any messages over the short-range wireless communication link. The timer duration may then be determined by a timer device comprised in the personal device.

In embodiments, the personal device is configured to establish the short-range wireless communication link with the mobile communications device while the short-range wireless communication link with the connection node has not been actively disconnected by the personal device.

In embodiments, the personal device is further configured to:
a. receive, from the assigned controller node via the established short-range wireless communication link with the connection node assigned to the personal device, a message containing data instructing the personal device to allow for the establishing of a short-range wireless communication link with a mobile communications device; and
b. establish a second simultaneous short-range wireless communication link with a mobile communications device communicatively coupled to a cellular mobile communications network.

In embodiments, the technology disclosed relates to a system including a local network comprising at least two network nodes and at least one personal device, the system is configured to assign a connection node among the at least two network nodes to each of the at least one personal device, wherein the assigned connection node is responsible for establishing a short-range wireless communication link with their respective personal device, and each of the at least one personal device is configured to transmit short-range wireless broadcast signals while having an active short-range wireless connection with a connection node assigned to the personal device, said system is further configured to:
a. establish a short-range wireless communication link between a personal device and a connection node assigned to said personal device;
b. obtain, by the connection node assigned to the personal device, signal strength values and/or packet loss rates over the short-range wireless communication link;
c. determine, by the controller node assigned to the personal device, that a link condition of the short-range wireless communication link is poor based on at least one of the signal strength values and/or high packet loss rates obtained by the connection node; and, in response to the determining that the link condition is poor:
d. transmit, from the controller node assigned to the personal device via the established short-range wireless communication link between the connection node and the personal device, a message containing data instructing the personal device to allow for the establishing of a short-range wireless communication link with a mobile communications device connected to a cellular mobile communications network, and
e. establish a second simultaneous short-range wireless communication link between the personal device and a mobile communications device connected to a cellular mobile communications network.

In embodiments, the technology disclosed relates to a system where the assigned controller node is a different node from the assigned connection node, and the system is further configured to perform the following step before determining the link condition:
h. transmit, or forward, by the assigned connection node, the obtained signal strength values and/or packet loss rates to the assigned controller node.

The short-range wireless broadcast signals may then be transmitted by the personal device when the personal device is within communication range with at least one of the nodes of the self-organizing network do not contain a flag indicating that the personal device allows the establishing of a short-range wireless communication link with a mobile communications device connected to a cellular mobile communications network. In response to and/or following the determining that a short-range wireless communication link with a node of the self-organizing network is disconnected, e.g. expired timer period, or is poor, e.g. low signal strength values and/or high packet loss rates, the personal device adds a flag to the short-range wireless broadcast signals it transmits. The personal device may then enable establishing a second simultaneous short-range wireless communication link with a mobile communications device, e.g. a mobile communications device associated with the personal device, even though a short-range wireless communication link with a node of the self-organizing network has not been actively disconnected.

In certain embodiments, only a mobile communications device which is associated with the personal device are allowed to establish a connection with the personal device, i.e. other mobile communications devices which are not associated with the personal device are not allowed to establish a connection to the personal device. According to these embodiments and in connection with transmitting at least one the short-range wireless broadcast signal containing a flag, the personal device may then be configured to enable it to establish a short-range wireless communication link with a mobile communications device associated with it, whereas the personal device is not enabled to establish a short-range wireless communication link with other mobile communications devices which are not associated with the personal device.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal;
b. receiving, by at least one node of the at least two network nodes, the at least one short-range wireless broadcast signal;
c. assigning one node among the at least two network nodes to be controller node for the personal device;
d. assigning a connection node for establishing a short-range wireless communication link between the connection node and the personal device;
e. establishing, by the assigned connection node, a first short-range wireless communication link between the personal device and the connection node;
f. transmitting, from the personal device, at least one short-range wireless broadcast signal containing a flag indicating that the personal device allows the establishing of a short-range wireless communication link with a mobile communications device connected to a cellular mobile communications network; and
g. establishing a second short-range wireless communication link between the personal device and the mobile communications device connected to a cellular mobile communications network.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal;
b. receiving, by at least one node of the at least two network nodes, the at least one short-range wireless broadcast signal;
c. assigning one node among the at least two network nodes to be controller node for the personal device;
d. assigning a connection node for establishing a short-range wireless communication link between the connection node and the personal device;
e. establishing, by the assigned connection node, a first short-range wireless communication link with the personal device;
f. transmitting, from the personal device, at least one short-range wireless broadcast signal containing a flag indicating that the personal device allows the establishing of a short-range wireless communication link with a mobile communications device associated with the personal device and which is connected to a cellular mobile communications network; and
g. establishing a second short-range wireless communication link between the personal device and the mobile communications device associated with the personal device and connected to a cellular mobile communications network.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal;
b. receiving, by at least one node of the at least two network nodes, the at least one short-range wireless broadcast signal;
c. assigning one node among the at least two network nodes to be controller node for the personal device;
d. assigning a connection node for establishing a short-range wireless communication link between the connection node and the personal device;
e. establishing, by the assigned connection node, a short-range wireless communication link with the personal device;
f. transmitting, from the personal device and via the established short-range wireless communication link, a message to the connection node;
g. receiving, by the connection node, the message, wherein the message contains data including at least one of status data, sensor data and event data associated with the personal device;
h. transmitting, from the assigned connection node to the controller node for the personal device, data including the at least one of status data, sensor data and event data associated with the personal device;
i. transmitting, from the personal device, at least one short-range wireless broadcast signal containing a flag indicating that the personal device allows the establishing of a short-range wireless communication link with a mobile communications device connected to a cellular mobile communications network; and
j. establishing a short-range wireless communication link between the personal device and the mobile communications device connected to a cellular mobile communications network.

In embodiments, the technology disclosed relates to a method in a system including a network comprising at least two network nodes and at least one personal device with short-range wireless communication capabilities, the method comprising:
a. transmitting, from a personal device, at least one short-range wireless broadcast signal;
b. receiving, by at least one node of the at least two network nodes, the at least one short-range wireless broadcast signal;
c. assigning one node among the at least two network nodes to be controller node for the personal device;
d. assigning a connection node for establishing a short-range wireless communication link between the connection node and the personal device;
e. establishing, by the assigned connection node, a short-range wireless communication link with the personal device;
f. transmitting, from the personal device and via the established short-range wireless communication link, a message to the connection node;
g. receiving, by the connection node, the message, wherein the message contains data including at least one of status data, sensor data and event data associated with the personal device;
h. transmitting, from the assigned connection node to the controller node for the personal device, data including the at least one of status data, sensor data and event data associated with the personal device;
i. transmitting, from the personal device, at least one short-range wireless broadcast signal containing a flag indicating that the personal device allows the establishing of a short-range wireless communication link with a mobile communications device associated with the personal device and which is connected to a cellular mobile communications network; and
j. establishing a short-range wireless communication link between the personal device and the mobile communications device associated with the personal device and connected to a cellular mobile communications network.

In certain aspects, technology disclosed relates to methods and a system comprising at least two network nodes and a plurality of personal devices each configured to transmit at least one short-range wireless broadcast signal which is received by any of the at least two network nodes within short-range wireless communication range with the respective personal device. In embodiments, the transmission of the at least one short-range wireless broadcast signal is performed while an established communication connection or link between the personal device and one of the network nodes which is the assigned connection node for the respective personal device is active. The transmission of broadcast signals may be performed in parallel with or while the personal device is transmitting messages via the established connection or link.

In embodiments, the personal devices are adapted to be worn by individuals, e.g. wristbands, and are configured to transmit short-range wireless broadcast signals while a short-range wireless connection (communication link) between the personal device and a node of the network of nodes is active. The controller node assigned to a personal device may then also be configured to take the decision to initiate a handover of the personal device to a mobile communications device in response to both no or low received signal strength values for the broadcast signals forwarded by other nodes and that the measured signal strength values and/or packet loss rates for the established short-range wireless communication link with the connection node assigned to the personal device is indicating poor link conditions.

According to aspects and certain embodiments of the technology disclosed, data or information contained in the at least one short-range wireless broadcast message received by the connection node via the established communication connection or link is forwarded via an IP-based network to the assigned controller node for the personal device The controller node, which has sole responsibility for the personal device, may be further configured to use data forwarded from the assigned connection node and/or data associated with the personal device which is received from the other nodes to take actions or make decisions on behalf of the personal device.

According to aspects and certain embodiments of the technology disclosed, the controller node assigned to a certain personal device may be configured to collect signal strength values of broadcast signals (transmitted from a certain personal device) from the other nodes of the network. The assigned controller node for the personal device may then be further configured to determine, based on the collected signal strength values, which of the nodes, including the controller node itself, should be the connection node for the personal device.

In aspects, the technology disclosed relates to a system including a network comprising at least two network nodes and a plurality of personal devices with short-range wireless communication capabilities. The system is configured to assign a connection node among the at least two network nodes to each of the plurality of personal devices. The connection node is responsible for establishing a short-range wireless communication link with the personal device it is assigned connection node for.

In embodiments, the controller node currently assigned to a personal device is configured to transmit alert state data and/or alarm data to a local or remote processing arrangement, e.g. backend system, based on received sensor data and/or event data. The local or remote processing arrangement may then be configured to set an alarm or determine whether to set an alarm based on the received alert state data and/or alarm data by informing and/or transmitting an alarm to the staff at the facility, e.g. send an alarm message to the mobile communication devices belonging to the staff members at the facility.

In embodiments, the local or remote processing arrangement may be configured to notify a mobile application program (APP) running on a mobile communication device, e.g. a mobile communication device trusted by, or associated with the personal device, in response to and/or based on the received alert state data and/or alarm data. The local or remote processing arrangement may then be configured to notify a mobile application program (APP) running on the mobile communication device in response to and/or based on the received alert state data and/or alarm data.

The user of the mobile communication device may belong to a staff member working at the facility where the SON network is installed or a family member and/or relative of the wearer of the personal device. The user of the mobile communication device may then have downloaded a mobile APP for an alert and/or alarm monitoring service associated with the SON network of the technology disclosed. The notification may be transmitted from the local or remote processing arrangement in response to and/or based on the received alert state data and/or alarm data and may then be received by the mobile communication device and presented in the mobile APP and/or display of the mobile communication device. The mobile communication device may belong to a staff member or a family member and/or relative of the wearer of the personal device, e.g. wristband, and may be listed and associated with the personal device and/or wearer in a memory storage of the local or remote processing arrangement, or a database or memory storage connected to or communicatively coupled to the local or remote processing arrangement.

In embodiments, the controller node currently assigned to a personal device is configured to transmit alert state data and/or alarm data to a local or remote processing arrangement, e.g. backend system, based on received sensor data and/or event data. The local or remote processing arrangement may then be configured to set an alarm or determine whether to set an alarm based on the received alert state data and/or alarm data by informing and/or transmitting an alarm to the staff at the facility, e.g. send an alarm message to the mobile communication devices belonging to the staff members at the facility.

In embodiments, each of the network nodes of the node network is configured to first measure signal strengths of short-range wireless broadcast signals received from a personal device and then automatically transmit or forward data indicating the measured signal strengths to the identified controller node responsible for the personal device transmitting the short-range wireless broadcast signals.

In embodiments, the controller node currently assigned to a personal device is configured to determine a position and/or a location for their respective personal device at least partly based on the received data indicating the measured signal strengths.

In embodiments, the controller node for the respective personal device is configured to set an alert state or alarm data based on received sensor data and/or event data and also determine a position and/or a location for their respective personal device at least partly based on the received data indicating the measured signal strengths.

In embodiments, the controller node for the respective personal device is configured to transmit alert state data and/or alarm data to a local or remote processing arrangement, e.g. backend system, based on received sensor data and/or event data and also determine a position and/or a location for their respective personal device at least partly based on the received data indicating the signal strengths of short-range wireless broadcast signals measured by the network nodes within short-range wireless communication range with the personal device transmitting the broadcast signals. The local or remote processing arrangement may then set an alarm or determine whether to set an alarm based on the received alert state data and/or alarm data by informing and/or transmitting an alarm to the staff at the facility where the alarm includes the determined position and/or location, e.g. send an alarm message to the mobile communication devices belonging to the staff members at the facility.

In embodiments, the controller node for the respective personal device is configured to set or change a health state for the individual wearing a personal device at least partly based on received sensor data obtained by and/or originating from at least one medical sensor of the personal device and also determine a position and/or a location for their respective personal device at least partly based on the received data indicating the measured signal strengths.

In embodiments, the determining of a position and/or location for the individual wearing the personal device by the controller node may be based on the received signal strength indication (RSSI) of short-range wireless broadcast signals transmitted by the personal device and which are received by the network nodes within short-range communication range with the personal device. The network nodes within short-range communication range then measure the RSSI of short-range wireless broadcast signals and forwards the RSSI to the controller node for the personal device. If the RRSI of short-range wireless broadcast signals received by more than one network node is determined, the location of the personal device may be more reliably determined based on e.g. triangulation. Machine learning may also be used for this determination. The measured RSSI values of short-range wireless broadcast signals transmitted by a personal device may be used by the controller node assigned to a personal device for determining the current position and/or location for the personal device in connection with transmitting position or location data together with alert state data or when setting an alarm for the individual wearing the personal device. Thus, the decision by the controller node for a particular personal device to transmit alert data or set an alarm for the individual may be based on broadcast signal strength values measured and received from other nodes within short-range communication range with the personal device (including the current controller node and the current connection node for the personal device if any of these two nodes are within short-range wireless communication range with the personal device to receive the broadcast signals).

In aspects, the technology disclosed relates to a method in a system including a network comprising network nodes and a personal device with short-range wireless communication capabilities, the method comprising:
a. assigning a network node to be controller node for the personal device;
b. assigning, by the controller node, a connection node for establishing a short-range wireless communication link between the connection node and the personal device;
c. establishing, by the assigned connection node, a short-range wireless communication link with the personal device;
d. transmitting, from the personal device and via the established short-range wireless communication link, a message to the connection node;
e. receiving, by the connection node, the message, wherein the message contains data associated with the personal device;
f. forwarding, from the assigned connection node to the controller node for the personal device, the message or data to the assigned controller node for the personal device; and
g. transmitting, from the personal device, a short-range wireless broadcast signal, wherein the short-range wireless broadcast signal is transmitted while the short-range wireless communication link between the connection node and the personal device is active, and wherein the short-range wireless broadcast signal is received or detected by any network node currently within short-range communication range with the personal device.

In aspects, the technology disclosed relates to a method in a system including a network comprising at least two network nodes and a plurality of personal devices with short-range wireless communication capabilities, the method comprising:
a. assigning, e.g. automatically by the at least two network nodes and based on a common decision model which is known to the at least two network nodes, one node among the at least two network nodes to be the controller node for a personal device;
b. assigning a connection node for establishing a short-range wireless communication link between the connection node and the personal device;
c. establishing, by the assigned connection node, a short-range wireless communication link with the personal device;
d. transmitting, from the personal device to the connection node via the established short-range wireless communication link, a message containing at least one of status data, sensor data and event data associated with the personal device;
e. receiving, by the connection node assigned to the personal device, the message;
f. transmitting, from the assigned connection node to the controller node for the personal device, data including the at least one of status data, sensor data and event data associated with the personal device; and
g. transmitting, from the personal device, at least one short-range wireless broadcast signal, wherein the at least one short-range wireless broadcast signal is transmitted while the short-range wireless communication link between the connection node and the personal device is active.

In aspects, the technology disclosed relates to a method in a system including a network comprising network nodes and a personal device with short-range wireless communication capabilities, the method comprising:
a. assigning a network node to be controller node for the personal device;
b. assigning, by the controller node, a connection node for establishing a short-range wireless communication link between the connection node and the personal device;
c. establishing, by the assigned connection node, a short-range wireless communication link with the personal device;
d. transmitting, from the personal device and via the established short-range wireless communication link, a message to the connection node;
e. receiving, by the connection node, the message, wherein the message contains data associated with the personal device;
f. forwarding, from the assigned connection node to the controller node for the personal device, the message or data to the assigned controller node for the personal device; and
g. transmitting, from the personal device, a short-range wireless broadcast signal, wherein the short-range wireless broadcast signal contains data associated with the personal device and is transmitted while the short-range wireless communication link between the connection node and the personal device is active, and wherein the short-range wireless broadcast signal is received or detected by any network node currently within short-range communication range with the personal device; and
h. forwarding, by any and all network nodes currently within short-range communication range with the personal device and to the controller node for the personal device, at least one of data extracted from the short-range wireless broadcast signal and measured, by each of the network nodes received the broadcast signal, respective signal strengths of the received short-range wireless broadcast signal.

In embodiments, the at least one of status data, sensor data and event data associated with the personal device is provided, e.g. automatically provided, to the controller node by forwarding the received message from the connection node or by transmitting, from the connection node, a message including data corresponding to, or at least partly based on, the received at least one of status data, sensor data and event data associated with the personal device.

In embodiments, the message is forwarded or transmitted via an IP-based network connecting the network nodes.

In embodiments, the IP-based network is a separate network and uses a different communication protocol from the communication protocol used for the established short-range wireless communication link between the connection node and the personal device.

In embodiments, the established short-range wireless communication link between the connection node and the personal device is a bidirectional short-range wireless communication link.

In embodiments, the message from the connection node to the controller node and the at least one short-range wireless broadcast signal transmitted from the personal device are containing data indicating the same sensor data and/or event data.

In embodiments, the message from the connection node to the controller node is containing data indicating at least one of the activation of an input means of the personal device, data indicating a trigger event and sensor data indicating a health state and/or alert state for the personal device and/or the individual wearing the personal device.

In embodiments, the at least one short-range wireless broadcast signal transmitted from the personal device is containing data indicating at least one of the activation of an input means of the personal device, data indicating a trigger event and sensor data indicating a health state and/or alert state for the personal device and/or the individual wearing the personal device.

In embodiments, the message from transmitted from the connection node is containing sensor data and/or event data, and the method is further comprising:
a. determining, by the controller node and at least partly based on received sensor data and event data associated with the personal device, a specific alert state and/or a health state for the individual wearing the personal device.

In embodiments, the message transmitted from the connection node is containing sensor data and/or event data, and the method is further comprising:
b. transmitting, from the controller node to a local or remote processing arrangement, at least one of the sensor data and/or event data and data indicating a changed health state and/or alert state for the individual wearing the personal device.

In embodiments, the message transmitted from the connection node is containing sensor data and/or event data, and the method is further comprising:
c. transmitting, from the controller node, the sensor data and/or event data associated with the personal device to a local or remote processing arrangement; and
d. determining, by the local or remote processing arrangement, the current health state and/or alert state for the individual wearing the personal device based on the sensor data and/or event data, wherein processing of the sensor data for determining the current health state and/or alert state is performed by the local or remote processing arrangement using a trained machine learning algorithm.

In embodiments, the method is further comprising:
e. determining, by the local or remote processing arrangement, a changed health state and/or alert state for the individual wearing the personal device controller node, wherein the determining is at least partly based on the received sensor data and event data associated with the personal device is indicating a specific alert state and/or a health state for the individual wearing the personal device; and
f. transmitting, from the controller node to a local or remote processing arrangement, data indicating the determined changed health state and/or alert state for the individual wearing the personal device.

In embodiments, the method is further comprising:
g. setting, by the local or remote processing arrangement, an alarm for the personal device and/or individual wearing the personal device, wherein the setting of an alarm is at least partly based on the determining of a changed health state and/or alert state for the individual wearing the personal device.

In embodiments, the method according to the technology disclosed is comprising:
a. receiving, by at least one network node within short-range wireless communication range with the personal device, at least one short-range wireless broadcast signal;
b. measuring, by each of the at least one network node within short-range wireless communication range with the personal device, the signal strengths of the at least one short-range wireless broadcast signal;
c. transmitting, from each of the at least one network node within short-range wireless communication range with the personal device and to the controller node, at least one message containing data indicating the respective signal strengths of the at least one short-range wireless broadcast signal measured by the respective at least one network node within short-range wireless communication range with the personal device; and
d. receiving, by the controller node for the personal device, at least one message containing data indicating the respective signal strengths, wherein a message is received from the respective of the at least one network node and is indicating the respective signal strengths measured by the respective at least one node within short-range wireless communication range with the personal device.

In embodiments, the method is further comprising:
h. determining, by the controller node and at least partly based on the data indicating measured signal strengths, the current position and/or location for the personal device and/or the individual wearing the personal device.

In embodiments, the determining of the current position and/or location for the personal device and/or the individual wearing the personal device is in addition based on sensor data obtained by a motion sensor and/or pressure sensor of the personal device.

In embodiments, the method is further comprising:
i. transmitting, from the controller node to a local or remote processing arrangement, data indicating the measured signal strengths for the respective at least one node; and
j. determining, by the local or remote processing arrangement, the current position and/or location for the personal device and/or the individual wearing the personal device, wherein the determining is at least partly based on received data indicating measured signal strengths for the respective at least one node.

In embodiments, the method is further comprising:
k. obtaining, by at least one of a motion sensor and a pressure sensor of the personal device, sensor data indicating a pressure and/or a movement associated with the personal device and/or the individual wearing the personal device; and
l. transmitting, from the personal device to the connection node via the established short-range wireless communication link, data indicating the sensor data obtained by the personal device;
m. receiving, by the connection node, data indicating the sensor data obtained by the personal device;
n. forwarding, by the connection node to the controller node, data indicating the sensor data obtained by the personal device; and
o. determining, by the controller node, the current position and/or location for the individual earing the personal device, wherein the determining of the current position and/or location is at least partly based on the sensor data obtained by a motion sensor and/or a pressure sensor of the personal device.

In embodiments, the message from the connection node is containing sensor data and/or event data, and the method is further comprising:
p. transmitting, from the controller node, the sensor data and/or event data associated with the personal device to a local or remote processing arrangement; and
q. determining, by the local or remote processing arrangement, the current health state and/or alert state for the individual wearing the personal device based on the sensor data and/or event data, wherein the processing of the sensor data for determining the current health state and/or alert state is performed by the local or remote processing arrangement using a trained machine learning algorithm.

In embodiments, the method is further comprising:
r. determining, by the controller node, a changed health state and/or alert state for the individual wearing the personal device controller node at least partly based on received sensor data and event data associated with the personal device is indicating a specific alert state and/or a health state for the individual wearing the personal device; and
s. transmitting, from the controller node to a local or remote processing arrangement, data indicating a changed health state and/or alert state for the individual wearing the personal device.

The technology disclosed provides a solution for acting on an identified event by transmitting (redundant) event data, e.g. event/alarm data which is triggered by pressing a button on a wristband or triggered by voice activation of a voice activation means of the personal device, both via an established short-range wireless connection with one of the nodes and via at least one short-range wireless broadcast signal or message, e.g. Bluetooth broadcast signals or messages, from the personal device and where the short-range wireless broadcast signal are received by the network nodes within short-range wireless communication range with the personal device.

In embodiments, the technology disclosed provides a solution for fast response to obtained sensor data or an identified event, e.g. health sensor data indicating a health state, e.g. a deteriorated health condition, of the individual wearing the personal device or the event of pressing an alarm button on a personal device such as a wristband. The fast response solution according to the technology disclosed is achieved by the transmission of sensor data and/or event data indicating health sensor data, e.g. obtained by a medical sensor, or the occurrence of an event such as the pressing an alarm button on the personal device or activating an voice activation means, both via an established short-range wireless connection with one of the network nodes and via short-range wireless broadcast transmissions, e.g. via Bluetooth, from the personal device and which are received by those network nodes which are within short-range wireless communication range, e.g. Bluetooth range, with the respective personal device transmitting the broadcast signals.

In embodiments, the technology disclosed provides a solution for dynamically and accurately determining the indoor position or location for a personal device by measuring, by each of the network nodes which are within short-range communication range with the personal device, the signal strength of short-range wireless broadcast signals, e.g. Bluetooth broadcast signals, received from the personal device.

In embodiments, the controller node for the personal device may then receive, via the IP-based network, data and/or information representing or reflecting the measured signal strengths from each of the plurality of nodes within short-range communication range with the personal device and, based on the data and/or information (including signal strengths measured by the current controller node itself and/or the current connection node for the personal device if any of these nodes are within short-range wireless communication range with the personal device to receive the broadcasts), determine the current position or location for the personal device.

In certain embodiments, the controller may in addition use other obtained sensor data, e.g. sensor data obtained from the movement sensor of the personal device, e.g. an accelerometer, to determine and/or calculate the current position and/or location for the personal device.

In certain embodiments, the controller node may forward the obtained data and/or information representing or reflecting the measured signal strengths received from the network nodes within short-range communication range with the personal device, and/or other movement sensor data obtained, to a locally-installed or remote processing arrangement such as a backend system and network which determines the current position or location for the personal device.

In certain embodiments, the current position or location, e.g. room, for the personal device may at least partly be determined by a trained machine learning algorithm used by the controller node or a locally-installed or remote processing arrangement such as a backend system communicatively coupled to the controller node, since machine learning is an efficient way of evaluating complex signals in situations where large amounts of data is collected. The trained machine learning algorithm may then use at least the signal strength received from the nodes within short-range wireless communication range (including the current controller node and the current connection node for the personal device if any of these two nodes are within short-range wireless communication range with the personal device to receive the broadcasts) and/or sensor data detected by a movement sensor of the personal device to determine the current position or location for the personal device.

In aspects, the technology disclosed relates to a method for a system comprising at least two or at least two network nodes configured to share data with the other nodes via an IP-based network and a plurality of personal devices each with Bluetooth communication capabilities, the method comprising the steps of:
a. establishing, by a connection node assigned to a personal device, a short-range wireless communication link between the connection node and the personal device, where the short-range wireless communication link is the only current communication link between the personal device and the network of nodes;
b. transmitting, from the personal device, data via the established short-range wireless communication link to the connection node; and
c. transmitting, from the personal device, short-range wireless broadcast messages, where the short-range wireless broadcast messages are transmitted while the personal device is transmitting data over and/or has the established short-range wireless communication link with the connection node assigned to the personal device.

In aspects, the technology disclosed relates to a method for a system comprising network nodes configured to share data with the other nodes via an IP-based network and a plurality of personal devices each with Bluetooth communication capabilities, the method comprising the steps of:
a. assigning, e.g. automatically by the nodes of the system, only one single controller node among the network nodes to have the sole responsibility for a personal device;
b. assigning, by the controller node, only one single connection node for the personal device, where the controller node is sole responsible for assigning connection node for the personal device;
c. establishing, by the assigned connection node, a short-range wireless communication connection link between the connection node and the personal device, where the short-range wireless communication connection link is the only current connection link between the personal device and the network of nodes;
d. transmitting, from the personal device, data via the established short-range wireless communication connection link to the connection node; and
e. transmitting, from the personal device, short-range wireless broadcast messages, where the short-range wireless broadcast messages are transmitted while the personal device is transmitting data over the established short-range wireless communication link with the connection node.

In aspects, the technology disclosed relates to a method for a system comprising at least two network nodes configured to share data with the other nodes via an IP-based network and a plurality of personal devices with Bluetooth communication capabilities, the method comprising:
a. assigning, e.g. by the nodes of the system using a common decision model which is known to all of the at least two network nodes, only one single controller node among the at least two network nodes to have the sole responsibility for the personal device;
b. assigning, by the assigned only one single controller node, only one single connection node for the personal device, wherein the controller node is sole responsible for assigning the only one single connection node to the personal device;
c. establishing, by the assigned only one single connection node, a Bluetooth connection between the only one single connection node and the personal device, wherein the Bluetooth connection is the only currently established Bluetooth connection from the network to the personal device;
d. transmitting, from the personal device, data via the established Bluetooth connection to the only one single connection node; and
e. transmitting, from the personal device, Bluetooth broadcast signals, wherein the Bluetooth broadcast signals are transmitted while the personal device has the established Bluetooth connection with the only one single connection node.

In embodiments, the technology disclosed relates to a method for a system comprising at least two network nodes and a plurality of personal devices with Bluetooth communication capabilities, the method comprising:
a. assigning, e.g. by the nodes of the system using a common decision model which is known to all of the at least two network nodes, only one single controller node among the at least two network nodes to have the sole responsibility for the personal device;
b. assigning, by the assigned only one single controller node, only one single connection node for the personal device, wherein the controller node is sole responsible for assigning the only one single connection node to the personal device;
c. establishing, by the assigned only one single connection node, a Bluetooth connection between the only one single connection node and the personal device, wherein the Bluetooth connection is the only current Bluetooth connection between the at least two network nodes and the personal device;
d. identifying, by the personal device, at least one event associated with the personal device or the user or wearer of the personal device;
e. transmitting, from the personal device, event data representing, or adapted to be used for identifying, the at least one event via the established Bluetooth connection to the only one single connection node;
f. transmitting or forwarding, from the connection node and following or in response to receiving the event data by the connection node, event data representing, or adapted to be used for identifying, the at least one event to the controller node;
g. transmitting, from the personal device, event data representing, or adapted to be used for identifying, the same at least one event in at least one Bluetooth broadcast message, wherein the at least one Bluetooth broadcast message is transmitted concurrently or in parallel with the transmission of event data via the established Bluetooth connection and while the personal device has the established Bluetooth connection with the only one single connection node;
h. receiving, by a plurality of network nodes within Bluetooth broadcast range with the personal device, the at least one Bluetooth broadcast message containing event data representing, or adapted to be used for identifying, the at least one event; and
i. transmitting, from at least one network node within Bluetooth broadcast range with the personal device, event data representing, or adapted to be used for identifying, the at least one event to the controller node.

In embodiments, the technology disclosed relates to a method for a system comprising at least two network nodes configured to share data with the other nodes via an IP-based network and a plurality of personal devices with Bluetooth communication capabilities, the method comprising:
a. assigning only one single controller node among the at least two network nodes to have the sole responsibility for the personal device;
b. assigning, by the assigned only one single controller node, only one single connection node for the personal device, wherein the controller node is sole responsible for assigning the only one single connection node to the personal device;
c. establishing, by the assigned only one single connection node, a Bluetooth connection between the only one single connection node and the personal device, wherein the Bluetooth connection is the only currently established Bluetooth connection between the at least two network nodes constituting the IP-based network and the personal device;
d. identifying, by the personal device, at least one occurred event associated with the personal device;
e. transmitting, from the personal device, event data representing the at least one occurred event via the established Bluetooth connection to the only one single connection node;
f. transmitting or forwarding, from the connection node and in response to receiving the event data by the connection node, event data representing the at least one occurred event to the controller node via the IP-based network;
g. transmitting, from the personal device, event data representing the same at least one occurred event in at least one Bluetooth broadcast message, wherein the at least one Bluetooth broadcast message is transmitted concurrently or in parallel with the transmission of event data via the established Bluetooth connection and while the personal device has the established Bluetooth connection with the only one single connection node;
h. receiving, by a plurality of network nodes within Bluetooth broadcast range with the personal device, the at least one Bluetooth broadcast message containing event data representing the same at least one occurred event; and
i. transmitting, from at least one network node within Bluetooth broadcast range with the personal device, event data representing the at least one occurred event to the controller node via the IP-based network.

In embodiments, the technology disclosed relates to a method for determining the current position or location for a personal device of a system comprising at least two network nodes and a plurality of personal devices with Bluetooth communication capabilities, the method comprising:
a. assigning only one single controller node among the at least two network nodes to have the sole responsibility for the personal device;
b. assigning, by the assigned only one single controller node, only one single connection node for the personal device, wherein the controller node is sole responsible for assigning the only one single connection node to the personal device;
c. establishing, by the assigned only one single connection node, a Bluetooth connection between the only one single connection node and the personal device, wherein the Bluetooth connection is the only current Bluetooth connection between the at least two network nodes and the personal device;
d. transmitting, from the personal device, data via the established Bluetooth connection to the only one single connection node;
e. transmitting, from the personal device, data in at least one Bluetooth broadcast message, wherein the at least one Bluetooth broadcast message is transmitted concurrently or in parallel with the transmission of data via the established Bluetooth connection and while the personal device has the established Bluetooth connection with the only one single connection node;
f. receiving, by a plurality of network nodes within Bluetooth broadcast range with the personal device, the at least one Bluetooth broadcast message;
g. measuring, by the plurality of network nodes within Bluetooth broadcast range with the personal device, signal strength values for the received at least one Bluetooth broadcast message;
h. transmitting or forwarding, from the plurality of network nodes within Bluetooth broadcast range with the personal device, the measured signal strength values, or data representing or reflecting the measured signal strength values, to the controller node; and
i. determining, by the controller node, the current position or location for the personal device at least partly based on the received measured signal strength values separately received from the plurality of network nodes.

In embodiments, the technology disclosed relates to a method for determining the current position or location for a personal device of a system comprising at least two network nodes and a plurality of personal devices with Bluetooth communication capabilities, the method comprising:
a. assigning only one single controller node among the at least two network nodes to have the sole responsibility for the personal device;
b. assigning, by the assigned only one single controller node, only one single connection node for the personal device, wherein the controller node is sole responsible for assigning the only one single connection node to the personal device;
c. establishing, by the assigned only one single connection node, a Bluetooth connection between the only one single connection node and the personal device, wherein the Bluetooth connection is the only current Bluetooth connection between the at least two network nodes and the personal device;
d. transmitting, from the personal device, data via the established Bluetooth connection to the only one single connection node;
e. transmitting, from the personal device, data in at least one Bluetooth broadcast message, wherein the at least one Bluetooth broadcast message is transmitted concurrently or in parallel with the transmission of data via the established Bluetooth connection and while the personal device has the established Bluetooth connection with the only one single connection node;
f. receiving, by a plurality of network nodes within Bluetooth broadcast range with the personal device, the at least one Bluetooth broadcast message;
g. measuring, by the plurality of network nodes within Bluetooth broadcast range with the personal device, signal strength values for the received at least one Bluetooth broadcast message;
h. transmitting or forwarding, from the plurality of network nodes within Bluetooth broadcast range with the personal device, the measured signal strength values, or data representing or reflecting the measured signal strength values, to the controller node; and
j. determining, by the controller node or a locally-installed or remote processing arrangement communicatively coupled to the controller node, the current position or location for the personal device at least partly based on the received measured signal strength values separately received from the plurality of network nodes.

In embodiments, the technology disclosed relates to a method for performing handover to a mobile communications device and/or changing connection node for a personal device of a system comprising at least two network nodes and a plurality of personal devices with Bluetooth communication capabilities, the method comprising:
a. assigning only one single controller node among the at least two network nodes to have the sole responsibility for the personal device;
b. assigning, by the assigned only one single controller node, only one single connection node for the personal device, wherein the controller node is sole responsible for assigning the only one single connection node to the personal device;
c. establishing, by the assigned only one single connection node, a Bluetooth connection between the only one single connection node and the personal device, wherein the Bluetooth connection is the only current Bluetooth connection between the at least two network nodes and the personal device;
d. transmitting, from the personal device, data via the established Bluetooth connection to the only one single connection node;
e. transmitting, from the personal device, data in at least one Bluetooth broadcast message, wherein the at least one Bluetooth broadcast message is transmitted concurrently or in parallel with the transmission of data via the established Bluetooth connection and while the personal device has the established Bluetooth connection with the only one single connection node;
f. receiving, by a plurality of network nodes within Bluetooth broadcast range with the personal device, the at least one Bluetooth broadcast message;
g. measuring, by the plurality of network nodes within Bluetooth broadcast range with the personal device, signal strength values for the received at least one Bluetooth broadcast message;
h. transmitting or forwarding, from the plurality of network nodes within Bluetooth broadcast range with the personal device, the measured signal strength values, or data representing or reflecting the measured signal strength values, to the controller node; and
k. determining, by the controller node or a locally-installed or remote processing arrangement communicatively coupled to the controller node, to perform handover to a mobile communications device, change connection node, or assign a new connection node, for the personal device, wherein the handover or change of connection node is at least partly based on the received measured signal strength values separately received from the plurality of network nodes.

### BRIEF DESCRIPTION OF DRAWINGS

Preferred embodiments of a system according to the technology disclosed will be described more in detail below with reference to the accompanying drawings wherein:

FIG. 1 schematically shows an example of facilities where a system comprising a number of network nodes have been installed, in accordance with one or more embodiments described herein.

FIG. 2 is a schematic illustration of a system for monitoring an individual within predetermined facilities.

FIG. 3 is an example flow diagram of a method for handover of a personal device between a network and a mobile communications device and the monitoring of an individual within predetermined facilities, in accordance with one or more embodiments described herein.

FIG. 4 schematically illustrates a method for monitoring an individual within predetermined facilities, in accordance with one or more embodiments described herein.

FIG. 5 schematically illustrates a method for handover of a personal device between a network and a mobile communications device and the monitoring of an individual within predetermined facilities, in accordance with one or more embodiments described herein.

FIG. 6 schematically illustrates a method for handover of a personal device between a network and a mobile communications device and the monitoring of an individual within predetermined facilities, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

In the drawings, similar details are denoted with the same reference number throughout the different embodiments. In the various embodiments of the system, e.g. monitoring system, according to the technology disclosed, the different subsystems are denoted. The “boxes”/subsystems shown in the drawings are by way of example only and can within the scope of the technology disclosed be arranged in any other way or combination.

In aspects, the technology disclosed relates to methods, a system, a network of nodes and a plurality of portable personal devices adapted to be worn by individuals and configured to transmit short-range wireless broadcast signals.

In embodiments, the technology disclosed relates to a personal device which is configured to first establish a short-range wireless communication link with a node of the self-organizing network and then enable the establishment of a short-range wireless communication link with a mobile communications device.

In embodiments, the personal devices are each configured to establish a short-range wireless communication link with the nodes of the self-organizing network and further configured to enable the establishment of a short-range wireless communication link with a mobile communications device associated with the respective personal device.

In embodiments, a self-organizing network is used and the personal device is further configured to enable a seamless handover procedure for handing over the personal device between a node of the self-organizing network and a mobile communications device associated with the personal device.

In embodiments, the technology disclosed relates to a method in a system including a network comprising at least two network nodes, at least one personal device and at least one mobile communications device connected and/or coupled to a cellular-type mobile communications network, the method comprising:
a. establishing a short-range wireless communication link between the personal device and a network node of the network;
b. enabling, by the personal device, the establishing of a short-range wireless communication link between the personal device and the at least one mobile communications device;
c. transmitting, from the personal device, at least one short-range wireless broadcast signal containing a flag indicating that the personal device allows the establishing of a short-range wireless communication link with the at least one mobile communications device; and
d. establishing a short-range wireless communication link between the personal device and one of the at least one mobile communications device.

In embodiments, the technology disclosed relates to a method in a system including a network comprising at least two network nodes and at least one personal device linked to and/or associated with at least one mobile communications device connected and/or coupled to a cellular-type mobile communications network, the method comprising:
a. establishing a short-range wireless communication link between the personal device and a network node of the network;
b. enabling, by the personal device, the establishing of a short-range wireless communication link between the personal device and the at least one mobile communications device linked to and/or associated with the personal device;
c. transmitting, from the personal device, at least one short-range wireless broadcast signal containing a flag indicating that the personal device allows the establishing of a short-range wireless communication link with the at least one mobile communications device linked to and/or associated with the personal device; and
d. establishing a short-range wireless communication link between the personal device and one of the at least one mobile communications device communicatively linked to and/or associated with the personal device.

In embodiments, the establishing of a short-range wireless communication link with a mobile communications device is in response to and triggered by a timer duration period of a timer of the personal device is exceeded without the personal device receiving any messages over the short-range wireless communication link or without receiving any message above a predefined signal strength threshold value.

In embodiments, the establishing of a short-range wireless communication link with a mobile communications device is followed by the step of receiving, by the personal device and from a mobile communications device, a connection request in the form of a broadcast signal for establishing a short-range wireless communication link with the mobile communications device.

In embodiments, the establishing of a short-range wireless communication link with a mobile communications device is in response to and triggered by a timer duration period of a timer (which indicates that a link condition for an established link is poor or that no messages are received over the link has been received) is exceeded and where the establishment of the link with the mobile communications device is initiated by the transmission of a short-range wireless broadcast signal containing a flag identifying the personal device when received by a mobile communications device.

In embodiments, the controller node assigned to the respective personal device by the self-organizing network is further configured to initiate handover to a mobile communications device and/or change the only one connection node for the personal device the controller node is responsible for at least partly based on data indicating the signal strength values and/or packet loss rates of Bluetooth signals transmitted over the established Bluetooth communication link between the personal device and the connection node assigned to the personal device and which are received and measured by the connection node.

In embodiments, the only one controller node assigned to the respective personal device is further configured to initiate handover to a mobile communications device and/or change the only one connection node for the personal devices the only one controller node is responsible for at least partly based on the data indicating the measured signal strength values of Bluetooth broadcast signals transmitted by the personal device and which are received and measured by the connection node and the other nodes of a self-organizing network.

In embodiments, the controller node which was assigned to the personal device according to a distributed decision model is configured to determine whether to change the connection node assigned to the personal device or initiate a handover procedure in which the personal device is establishing a second communication link with a mobile communications device and the established communication link with the connection node is actively or passively disconnected. The decision by the controller assigned to the personal device whether to change connection node or initiate a handover procedure to handover the personal device to a mobile communications device may be at least partly based on measurement data received from the connection node and at least one other node. The measurement data received by the controller node may then indicate signal strength values and/or packet loss rates for Bluetooth signals transmitted over the established communication with the connection node and Bluetooth broadcast signals transmitted by the personal device and which are received and measured by the at least one other node and, optionally, also the connection node currently assigned to the personal device.

In embodiments, the only one controller node assigned to the respective personal device is further configured to determine the current position of the personal device it is responsible for based on the data indicating the measured signal strength values of Bluetooth broadcast signals transmitted by the respective personal device and which are received and measured by the plurality of other nodes. The decision by the controller node whether to initiate handover of the personal device from the connection node to a mobile communications device may then be at least partly based on the determined current position for the personal device.

In embodiments, the technology disclosed provides a fast response yet robust solution for acting on an identified event by parallel and/or redundant data transmission of event data representing or reflecting the event via both an established short-range wireless connection with one of the nodes and via short-range wireless broadcast messages from the personal device to the nodes which are within short-range wireless communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In embodiments, the technology disclosed provides a solution for dynamically and accurately determining the indoor position or location for a personal device without any use of high energy-consuming GPS in the personal devices or beacons, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In aspects, the technology disclosed relates to a system including a network comprising at least two network nodes configured to share data with the other nodes via an IP-based network and a plurality of personal devices with Bluetooth communication capabilities, the plurality of personal devices are each assigned only one controller node among the at least two network nodes, wherein the only one controller node is responsible for assigning only one connection node among the at least two network nodes to be responsible for establishing a Bluetooth connection with the personal device the controller node is responsible for, and wherein the plurality of personal devices are each configured to be transmitting Bluetooth broadcast signals while having an established Bluetooth connection with their respectively assigned only one connection node.

In embodiments, the only one controller node assigned to the respective personal device is configured to assign and dynamically change which of the at least two network nodes is currently the only one connection node for their respective personal devices at least partly based on at least one of contents and received signal characteristics of at least one Bluetooth broadcast signal transmitted from the personal device and which is received by a plurality of other nodes than the controller node.

In embodiments, the only one controller node assigned to the respective personal device is configured to receive data via the IP-based network from a plurality of other nodes than the controller node, and wherein the data received via the IP-based network include data indicating the measured signal strength values of Bluetooth broadcast signals transmitted by the respective personal device and which are received and measured by the plurality of other nodes.

In embodiments, the system further comprises a locally-installed or remote processing arrangement such as a backend system and network configured to receive data indicating measured Bluetooth broadcast signal strength values for a personal device from the only one controller node responsible for the personal device, and wherein the locally-installed or remote processing arrangement, e.g. backend system and network, is further configured to determine the current position for the personal device based on the received Bluetooth broadcast signal strength values for the personal device and return the determined position to the only one controller node responsible for the personal device.

In embodiments, each of the plurality of personal devices is configured to transmit Bluetooth broadcast signals including alarm data in response to a trigger event determined by the respective personal device to have occurred, and wherein the alarm data is adapted to indicate the occurrence of the trigger event to any node receiving the Bluetooth broadcast signal and is transmitted by the respective personal device while having an established Bluetooth connection with their respectively assigned only one connection node.

In embodiments, each of the plurality of personal devices is further configured to, in addition, transmit data including redundant alarm data in response to the determined occurrence of the trigger event via the established Bluetooth connection with their respectively assigned only one connection node, and wherein the respectively assigned only one connection node is configured to, in response to receiving the alarm data via the established Bluetooth connection, at least one of transmit and forward data including alarm data via the IP-based network to the respectively assigned only one controller node, where the alarm data is adapted to indicate to the only one controller node that the trigger event has occurred.

In embodiments, the system comprises at least one mobile communication device and a backend system and network configured to receive alarm data indicating the occurred trigger event from the only one controller node responsible for the personal device which determined that the trigger event occurred, and wherein the backend system and network is further configured to transmit alarm data to the at least one mobile communication device in response to receiving the alarm data from the only one controller node.

In embodiments, the local or remote processing arrangement, e.g. backend system and network, is further configured to transmit the alarm data to the at least one mobile communication device via a network separate from the IP-based network used by the at least two network nodes to exchange data.

According to certain aspects and embodiments, the assignment of roles for the network nodes is associated with a specific individual personal device and is done on two levels, a control level through the assignment of a controller node and a connect level through the assignment of a connection node. The role assigned to a network node may be dynamically changed based on data obtained by the personal devices and/or the node network.

The system, e.g. monitoring system, comprising the node network of the technology disclosed is highly scalable and provides reliable and fast responses to obtained sensor data, event data and inputs from the monitored individuals, yet is robust in that each of the personal devices always has a reliable short-range wireless connection to the node network which is not disturbed or compete for short-range wireless bandwidth with other devices connected to the same node of the node network or with other mobile communications devices in the vicinity of the node network which are not associated with the personal device.

According to certain aspects and embodiments, the system and methods of the technology disclosed provides a scalable solution in the dynamic reconfiguration of the node networks on two levels, yet is robust and reliable in that each of the personal devices always is provided a reliable short-range wireless connection to the network and/or a reliable short-range wireless connection to a mobile communications device when outside communication range with the network. In embodiments, this is achieved by assigning a controller node to each personal device which is responsible for assigning a connection node to the personal device and dynamically changing connection node for the personal device, where the connection node is responsible for establishing a short-range wireless connection with the personal device and the personal device is configured to enable the establishment of a short-range wireless connection with a mobile communications device, e.g. associated with the personal device.

According to aspects of the technology disclosed, the other network nodes shares or forwards any received or obtained data or information, e.g. measured signal strength data and/or packet loss rates, status data, sensor data, event data or other types of input data, which is received from or associated with a certain personal device to the controller node for the personal device. This allows for redundancy and, thus, safer and more accurate decision-making by the controller node, e.g. handover decisions, and/or the backend system, as well as provides faster responses to events, e.g. the pressing of a button on the personal device, and obtained sensor data in that all data or information associated with a certain personal device is quickly available to the one controller node having the sole responsibility for taking actions and making decisions on behalf of the personal device, e.g. fast responses in terms of making a decision to set an alert, change a health state and/or change connection node for the personal devices.

According to aspects of the technology disclosed, there is only one controller node for each personal device and the system is configured so that each of the other network nodes shares or forwards any received or obtained data or information, e.g. measured signal strength data and/or packet loss rates, status data, sensor data, event data or other input data, associated with a certain personal device to the controller node for the personal device. This provides for redundancy and, thus, safer and more accurate decision-making by the controller node, e.g. handover decisions, and/or the backend system, as well as faster responses to events and obtained data in that all data or information associated with a certain personal device is received by the one controller node having the sole responsibility for making decisions and taking actions on behalf of the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

In certain embodiments, the current position or location for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically changing the connection node for a personal device, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine to change connection node for the personal device.

In various embodiments, the controller node may forward the obtained data and/or information representing or reflecting the measured signal strengths from each of the plurality of other nodes, and/or other movement sensor data obtained, to a backend network which determines that the connection node for the personal device should be changed.

In various embodiments, the controller node may in addition use other obtained sensor data, e.g. sensor data from a movement sensor of the personal device, e.g. an accelerometer, to determine that the connection node for the personal device should be changed.

In certain embodiments, the change of connection node for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine that the connection node for the personal device should be changed.

The systems and networks, e.g. piconets or SON, known in the art further does not disclose a system, e.g. monitoring system, comprising a node network and personal devices with broadcast capabilities which is self-organizing and scalable by dynamically assigning roles to the individual nodes, e.g. continuously and automatically assigning roles to the nodes, where each assignment of a role to a node relates to managing and control of only one of the personal devices. In particular, the systems and network solutions known in the art are silent to dynamically assigning and changing roles to the nodes on two levels, a control level where the node has the sole responsibility for a personal device and a connection level where the node is responsible for establishing a connection or link with the personal device.

The ad-hoc networks known in the art does not disclose or describe a node network where the nodes communicate internally via their IP addresses, but where the communication between personal devices and the network nodes takes place via a short-range wireless communication such as Bluetooth. This gives a much higher bandwidth capacity for the communication between the personal devices and the network nodes. This also enables the node network to be scalable and more distributed, since communication between network nodes can take place via the IP-based network connecting the nodes.

To address the identified problems with the prior art, the technology disclosed proposes a system, e.g. monitoring system, and node network for dynamically assigning roles to the nodes of the network on two levels, a control level and connect level.

The technology disclosed introduces a node network where the nodes communicate internally via their IP addresses, but where the communication between personal devices and nodes takes place via short-range wireless communication such as Bluetooth. This gives a better load balancing and much higher bandwidth capacity for the communication between the personal devices and the nodes. This also enables the node network to be more scalable and distributed, since communication between nodes can take place via an IP-based network.

The system, e.g. monitoring system, comprising the node network of the technology disclosed is highly scalable and provides for safe and fast responses to events, obtained data and the movements of the individuals, yet is robust in that each of the personal devices always has a reliable short-range wireless connection to the node network which is not disturbed or compete for short-range communication bandwidth with other devices connected to the same node of the node network.

In certain embodiments, the technology disclosed relates to embodiments of a handover procedure which includes the following process steps when the personal device, e.g. a wristband, is leaving a self-organizing network and is coming home to a self-organizing network, respectively:

Handover from a self-organizing network to a mobile device when the personal device is leaving the self-organizing network:
1. After being disconnected for x seconds, the personal device allows connections from mobile device(s). After a mobile device has connected to the personal device, the personal device enables a second simultaneous connection.

Handover from a mobile device to a self-organizing network when the personal device is coming home to the self-organizing network:
1. Self-organizing network connects to the personal device on the second connection.
2. On connection to the personal device, the personal device disconnects from the mobile device and disables the second connection.

In certain embodiments, the technology disclosed relates to embodiments of an even more graceful handover procedures including the following process steps when the personal device, e.g. a wristband, is leaving a self-organizing network and is coming home to a self-organizing network, respectively:

Graceful handover from a self-organizing network to a mobile device when the personal device is leaving the self-organizing network:
1. Self-organizing network starts to get a bad connection to the personal device based on signal strength and/or packet drops and there is no network node in the Self-organizing network that is closer to the current connection node (or receives better signal strength values) than the connected node.
2. The controller node tells the personal device to enable a second simultaneous connection from a mobile device.
3. If the mobile device connects to the personal device, the personal device drops the connection to the Self-organizing network, but will still allow it to reconnect when the Self-organizing network decides the connection is good enough for a reconnect.
4. If the mobile device does not connect to the personal device, or is not available, the personal device will stay connected to the Self-organizing network.

Graceful handover from a mobile device to a self-organizing network when the personal device is corning home to the self-organizing network:
1. Self-organizing network connects to the personal device on the second connection.
2. On connection to the personal device, the personal device disconnects from the mobile device and disables the second connection.

In aspects, the technology disclosed allows for faster and more accurate decision-making and improved load balancing within a network by providing methods and a system comprising personal devices and a network of nodes for dynamically assigning one controller node and one connection node for each personal device. In embodiments, the controller node for a personal device is dynamically assigned or changed based on a common decision model known to the network nodes and which may contain input values or parameters. The input values or parameters may be changed with the data distributed and shared between the network nodes. In certain embodiments of the technology disclosed, the consensus model as such with its input values or parameters may also be dynamically adjusted and changed over time.

In embodiments, the technology disclosed relates to a system comprising a node network which is self-organization by including a dynamic assignment of a node as uploader node to the internet or backend system.

In certain embodiments, the technology disclosed relates to a system comprising a node network which is self-organization by assigning a “stand-by” node to take over from the assigned node, e.g. a stand-by node which is ready to take over from an assigned controller node, an assigned connection node and/or an assigned uploader node.

In certain embodiments, the assignment of a stand-by controller node may use the same common decision model as used for the assignment of the controller node where the decision model may, or may not, use input or parameter data values obtained from the personal devices and/or nodes of the network to determine which of the at least two nodes is going to be the stand-by node. The input or parameter data values may change the determining of which of the network nodes is going to be the stand-by controller node for the personal device.

In certain embodiments, the assignment, by the controller node, of a stand-by connection node to take over from the assigned connection node may be at least partly based on signal strengths of short-range wireless broadcast signals which are measured by a plurality of network nodes within short-range communication range with the personal device transmitting the short-range wireless broadcast signals. The assignment of stand-by connection node by the controller node may further use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

The faster and more accurate decision-making is at least partly achieved by the method of assigning, for each personal device, one controller node with sole responsibility for the personal device, where the controller node is provided with all data or information associated with the personal device the controller node is responsible for and which is received, obtained and/or measured by the network nodes.

The faster and more accurate decision-making may be further achieved by the controller node making all decisions for the personal device, e.g. the decision to set an alert or transmit alarm data or the assignment of connection node with which the personal device is establishing a short-range wireless communication link such as a Bluetooth connection. The connection node may then have an established connection with the personal device as long as the controller node considers it appropriate, e.g. as long as the signal strength measured by the connection node is above a certain threshold value and/or a signal strength measured by the connection node is higher than signal strengths measured by the other nodes of the network.

Improved load-balancing is achieved by having the controller node with sole responsibility for a personal device dynamically assigning and changing connection node for the personal device at least partly based on load-balancing factors such as the current number of established short-range wireless connections for a node, e.g. as compared to the number of connections for other nodes, to thereby distribute the amount of short-range wireless data transmitted locally.

In certain aspects, the system and self-organizing network of the technology disclosed separating the data traffic in the IP-based network traffic from the short-range wireless communication performed by the personal devices provides an improved solution over ad-hoc networks and many state of the art self-organizing networks in that the transmission of data between the nodes used for configuring and self-organizing the network nodes, e.g. data used for assigning different roles to the nodes, is performed via the IP-based network and not via the short-range wireless protocol the personal device are using for providing the network nodes with data, thereby does not load, affect or interfere with the short-range wireless radio traffic, e.g. the Bluetooth radio traffic.

Benefits of the technology disclosed include that the system, self-organizing network and methods provide each of the personal devices with improved short-range wireless connections by continuously and dynamically changing which of the network nodes is the sole connection node having the only short-range wireless connection, e.g. Bluetooth connection, to a personal device at least partly based on measured signal strengths for the currently established short-range wireless connection and/or short-range signal strengths measured by the individual nodes of the network.

Further benefits of the technology disclosed include that the controller node is provided with data associated with the personal device it is responsible for from the other nodes of the network in that all data or information associated with the personal device, or data or information representing or reflecting the obtained data or information, is forwarded from the other network nodes to the controller node, including the data received and measured by the currently assigned connection node for the personal device.

In embodiments, the technology disclosed provides a fast response yet robust solution for acting on an identified event by parallel redundant data transmission of event data representing or reflecting the event via both an established short-range wireless connection with one of the nodes and via short-range wireless broadcast messages from the personal device to the nodes which are within short-range wireless communication range.

Bluetooth is a short-range wireless technology standard that allows data to be exchanged between fixed and mobile devices. The technology uses short wavelength radio waves from 2.4 to 2.485 GHz. Unlike other forms of connectivity such as wi-fi or 4G, Bluetooth carries connections between devices and other devices as opposed to carrying data to and from the internet.

BLE stands for Bluetooth Low Energy and is a form of wireless communication designed especially for short-range communication. BLE is very similar to Wi-Fi in the sense that it allows devices to communicate with each other. However, BLE is meant for situations where battery life is preferred over high data transfer speeds. Wi-Fi uses multiple parts of the IEEE 802 protocol family and is designed to interwork seamlessly with its wired sibling Ethernet. Compatible devices can network through wireless access points to each other as well as to wired devices and the Internet. The different versions of Wi-Fi are specified by various IEEE 802.11 protocol standards, with the different radio technologies determining radio bands, and the maximum ranges, and speeds that may be achieved. Wi-Fi most commonly uses the 2.4 GHz (120 mm) UHF and 5 GHz (60 mm) SHF ISM radio bands; these bands are subdivided into multiple channels. Channels can be shared between networks but only one transmitter can locally transmit on a channel at any moment in time.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In various embodiments, the concurrent or parallel transmissions of event data representing the same at least one event from the personal device via both the established Bluetooth connection and via at least one Bluetooth broadcast message are triggered by at least one event identified by the personal device.

In embodiments, the at least one event identified by the personal device and which triggers the parallel and redundant transmissions of event data may be sensor data sensed or measured by a sensor of the personal device or may be triggered by activating, by the individual or object carrying or wearing the personal device, an input means of the personal device such as the event of pressing of a button, e.g. an alarm button, on the personal device or an event identified by voice activation using a voice activation means of the personal device.

In embodiments, the sensed or measured sensor data that triggers the parallel and redundant transmissions of event data representing the at least one event may include that the personal device identifies that a sensed or measured sensor data exceeds or under-runs at least one predefined threshold value, e.g. a predefined threshold temperature value such as a predefined threshold body temperature value of the individual or object carrying or wearing the personal device.

In embodiments, the sensor which sensed or measured sensor data triggers the parallel or redundant transmissions of event data from the personal device may be a location or position detector or sensor, e.g. an RFID means, for determining the current position or location of the individual carrying or wearing the personal device. The sensed or measured sensor data that triggers the parallel and redundant transmissions of event data representing the at least one event may then include that the personal device identifies that the individual or object carrying or wearing the personal device has changed his/her location or position by receiving or exchanging, e.g. wirelessly receiving or exchanging location or position data, from/with a wireless transmitter or transceiver. The received or exchanged location or position data may then be used by the personal device to determine that the personal device is in a certain position or location, e.g. a certain room, of a facility in which a monitored individual may be residing, e.g. a nursing home, where there may be a private space for the monitored individual, including e.g. a bedroom, as well as public spaces that are shared by a plurality of monitored individuals. The parallel or redundant transmissions of event data may also be at least partly based on a timer of the personal device is indicating a certain movement pattern of the individual wearing the personal device, and/or at least partly based on a timer of the personal device is indicating that a certain predefined time period has lapsed since the individual activated an input means of the personal device, e.g. that a certain predefined time period has lapsed since the individual pressed a button of the personal device.

In embodiments, the sensor which sensed or measured sensor data triggers the parallel or redundant transmissions of event data from the personal device may be a movement sensor such as an accelerometer for determining the movement speed and/or pattern of the individual carrying or wearing the personal device. The sensed or measured sensor data that triggers the parallel and redundant transmissions of event data representing the at least one event may then include that the personal device identifies that a sensed or measured sensor data exceeds or under-runs at least one predefined threshold value, e.g. a predefined threshold movement speed, a predefined threshold average movement distance per time unit and/or threshold movement pattern value of the individual or object carrying or wearing the personal device.

In embodiments, the sensor which sensed or measured sensor data triggers the parallel or redundant transmissions of event data from the personal device may be a health or medical sensor such a body temperature sensor for determining the body temperature of the wearer, or may include a blood pressure sensor, a heart rate sensor, a skin galvanometric level sensor etc.

In embodiments, the personal device may be a wristband and at least one sensor of the personal device may include at least one health or medical sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a temperature sensor for obtaining sensor data for determining the body temperature of the wearer, or may include a blood pressure sensor, a heart rate sensor, a skin galvanometric level sensor etc.

In embodiments, the technology disclosed provides a solution for dynamically and accurately determining the indoor position or location, e.g. current room, for a personal device without any use of high energy-consuming GPS in the personal devices or iBeacon technology, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

The controller node, or a local or remote processing arrangement receiving the measured signals strengths from the controller node (and optionally also sensor data obtained by a sensor of the personal device), may then be configured to determine the current position or location for the personal device by comparing the signal strengths of the broadcast signals measured by the other nodes (and the controller node) and then determine the position or location based on the comparison. In embodiments, the position or location of the node measuring the highest signal strength for the broadcast signal is determined to be the position or location for the personal device, or the position or location for the personal device may be determined or calculated using signals strengths measured by a plurality of nodes, e.g. by using triangulation, or the position or location for the personal device may be determined or calculated using at least one signals strength of a broadcast signal and sensor data obtained by a sensor of the personal device, e.g. a motion sensor or a pressure sensor.

In certain embodiments, the current position or location for the personal device may at least partly be determined by a trained machine learning algorithm used by the controller node or a backend system communicatively coupled to the controller node, since machine learning is an efficient way of evaluating obtained sensor data and event data, e.g. in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically and continuously changing the connection node for a personal device, by measuring, by each of the network nodes which are within short-range communication range with the personal device, the signal strength of short-range wireless broadcast messages, e.g. Bluetooth broadcast messages, received from the personal device.

In certain embodiments, the setting of an alarm or the change of health state and/or alert state for the personal device may at least partly be determined by a trained machine learning algorithm used by the controller node or a backend system communicatively coupled to the controller node, since machine learning is an efficient way of evaluating sensor data in situations where large amounts of data is collected. The trained machine learning algorithm may then use at least the signal strength values received from the nodes (including the current controller node and the current connection node for the personal device if any of these nodes are within short-range wireless communication range with the personal device to receive the broadcasts) and/or movement sensor data detected by the personal device to determine that the connection node for the personal device should be changed.

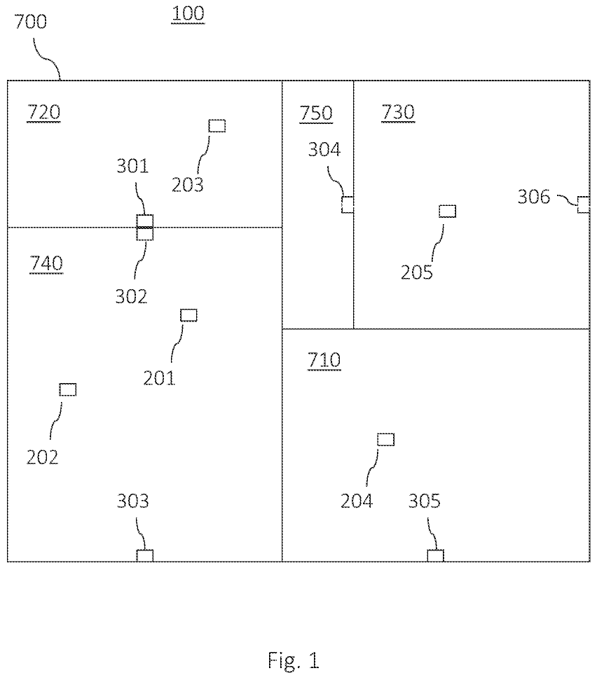

FIG. 1 schematically shows an example of a facility 700 where a system 100 comprising a number of network nodes (301, 302, 303, 304, 305, 306) has been installed, in accordance with one or more embodiments described herein. The illustrated system 100 comprises a plurality of personal devices (201, 202, 203, 204, 205) with short-range wireless communication capability. Such facility 700 may e.g. be an apartment where a monitored individual is residing. Such an apartment 700 may e.g. comprise a kitchen 710, a bathroom 720, a bedroom 730, a sitting room 740, and a hallway 750. The system 100 may in this case be used by relatives, home care companies or the like.

The facility 700 may also be other types of quarters where a monitored individual may be residing, such as e.g. a nursing home, where there may be a private space for the monitored individual, including e.g. a bedroom 730, as well as public spaces that are shared by a plurality of monitored individuals.

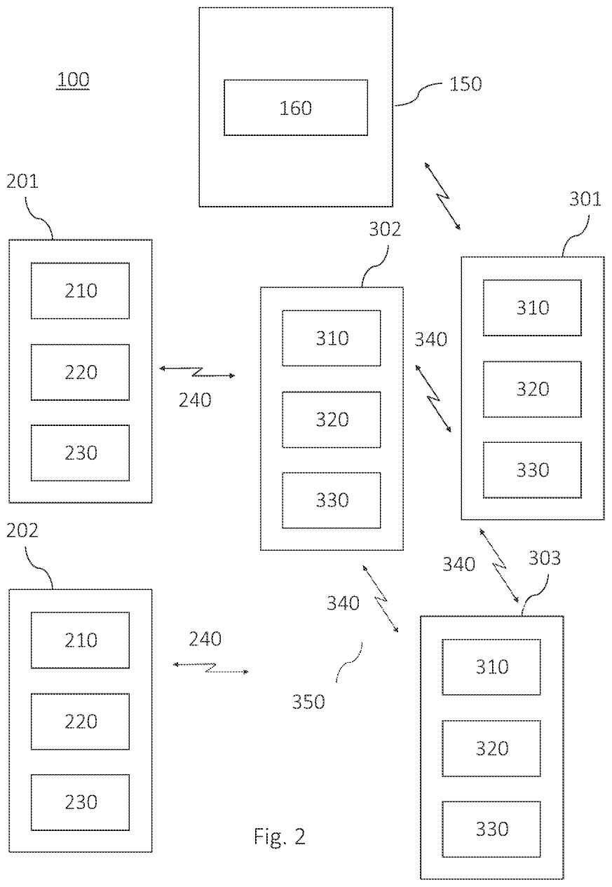

FIG. 2 is a schematic illustration of a system 100 for monitoring an individual within predetermined facilities 700, in accordance with one or more embodiments described herein. The illustrated system 100 comprises a plurality of personal devices (201, 202), a plurality of network nodes (301, 302, 303), and a processing arrangement 150, e.g. a locally-installed processing arrangement or a remote processing arrangement such as a backend system. The personal devices (201, 202) may be a wristband or other portable device that is carried or worn by an individual, e.g. a necklace or a clip worn by a patient in a nursing home. The illustrated personal devices (201, 202) comprise a processing device 210, at least one sensor and/or user input means 220, and a personal device communication interface 230. The at least one sensor may, for example, include a motion sensor, a temperature sensor, a health sensor such as a body temperature sensor, a pressure sensor and/or any other type of sensor and the user input means may comprise a button or voice input means that the individual can press or activate to set off an alarm and/or transmit event data indicating the occurrence of a certain event. The illustrated network nodes (301, 302, 303) comprise a node processing device 310, a storage means 320, and a node communication interface 330. The illustrated processing arrangement 150 comprises a processing device 160, which may e.g. be comprised in a web server. However, the system 100 does not necessarily comprise a processing arrangement 150.

Information regarding the layout of the facilities 700, such as the plan of the different rooms and the location of the node modules 300, is preferably stored in the storage means 320. The storage means 320 does not have to be comprised in the network node—it can be comprised in another part of the system such as a locally-installed processing arrangement or a remote processing arrangement such as a backend system, or be a separate device or module.

According to embodiments of the technology disclosed, the at least one processing device 310 of the network nodes (301, 302, 303) may be arranged to: receive status data, position data or signal strength values from another node of the network, or sensor data, input data or event data originating from the at least one sensor and/or user input means 220 in the personal device 200; determine whether the received data indicates a need for certain action associated with the personal device, e.g. changing health state for the individual wearing the personal device and/or alert state among a plurality of predetermined alert states. As an example, the alert states may comprise at least a FALL alert state, to be used if a probable fall has been detected for the monitored individual, and an OUT OF BED alert state, to be used if it has been detected that the monitored individual is probably getting out of bed.

In various embodiments, the at least one processing device 210, 310, 160 processing the sensor data, input data or event data may be the personal device processing device 210, the node processing device 310, or another processing device, such as a local or remote processing device 160. The at least one processing device may also be a combination of any number of processing devices, so that some of the processing takes place in one processing device and some of the processing takes place in one or more other processing devices. It is thus not necessary for all of the processing to take place in the same processing device.

If weighing factors are used, these weighing factors may be determined by a machine learning system, since this is an efficient way of evaluating complex signals in situations where large amounts of data is collected.

One way of making such a determination is to determine a probability that an alert should be set based on the received event data and/or sensor data, and a probability that the determined location fulfils the predetermined location condition, and then combine these probabilities using weighing factors, which may e.g. be determined by a machine learning system.

The same applies to other combinations, such as e.g. the determination of the position and/or location for the individual wearing the personal device by involving also signals from other sensors of the personal device such as a movement sensor 220, and/or the setting of alerts based also on activity states. These determinations may also be done by determining probabilities and using weighing factors to combine them, which may e.g. be determined by a machine learning system.

In embodiments, the determining of a position and/or location for the individual wearing the personal device by the controller node (301) may e.g. be based on the received signal strength indication (RSSI) of short-range wireless broadcast signals transmitted by the personal device and which are received by the network nodes within short-range communication range with the personal device. The network nodes within short-range communication range then measure the RSSI of short-range wireless broadcast signals and forwards the RSSI to the controller node for the personal device. If the RRSI of short-range wireless broadcast signals received by more than one network node is determined, the location of the personal device may be more reliably determined based on e.g. triangulation. Machine learning may also be used for this determination. The measured RSSI values of short-range wireless broadcast signals transmitted by a personal device may be used by the controller node assigned to a personal device for determining the current position and/or location for the personal device in connection with transmitting position or location data together with alert state data or when setting an alert or alarm for the individual wearing the personal device. Thus, the decision by the controller node for a particular personal device to set an alarm for the individual or to transmit alert state data or alarm data may be based on broadcast signal strength values measured and received from other nodes within short-range communication range with the personal device (including the current controller node and the current connection node for the personal device if any of these nodes are within short-range wireless communication range with the personal device to receive the broadcasts).

The personal devices 200 may also comprise other sensors 220, such as e.g. a pressure sensor, temperature sensor or medical sensor. It is known to use a pressure sensor in a personal device to detect a fall based on a change in pressure. However, a pressure sensor may also be involved in the determination of the location of the individual, e.g. when location data is transmitted together with health state data and/or alert state data to a local or remote processing arrangement, e.g. a backend system, configured to set an alert or an alarm. If the facilities 700 comprise several floors, and the node modules 300 are arranged in the ceilings or high up on the walls, the RSSI may give erroneous results due to the signals travelling between the floors. In this situation, a pressure sensor may be used to determine the floor on which the individual is located.

In order to improve the determination of the need for e.g. changing health state for the individual and/or setting an alert even further, the determination of the location may be improved by involving also signals from a movement sensor 220. The movement sensor 220 may e.g. be used as a pedometer, so that the signals from the movement sensor 220 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 220, it is likely that the determined location is incorrect. The at least one processing device 210, 310, 160 may thus be arranged to determine the location of the personal device 200 based also on an indication of the movement of the individual in relation to a previously determined location, based on sensor data from the at least one motion sensor 220.

The processing of the sensor data from the at least one sensor 220 may be done by a trained machine learning algorithm used by the controller node or a local or remote processing arrangement communicatively connected to the personal device. When many individuals are monitored using many personal devices 200, large amounts of sensor data is collected from the sensors 220 in these personal devices 200. Very efficient evaluation of this sensor data may be done using machine learning, in order to determine a suitable machine learning algorithm.

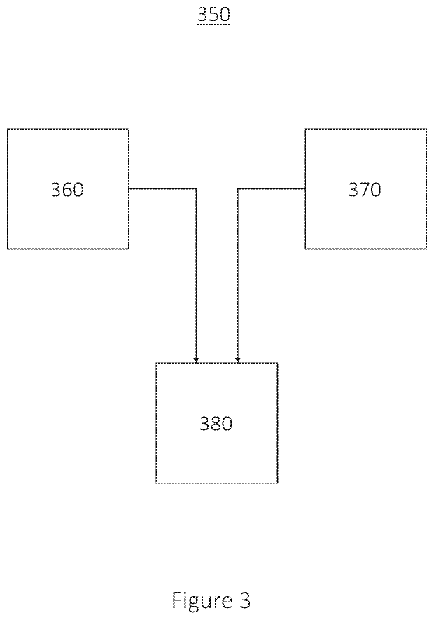

FIG. 3 is an example flow diagram of a method for handover of a personal device from a network to a mobile communications device and the monitoring of an individual within and outside predetermined facilities in order to determine whether there is a need for responding to status data, sensor data and/or event data received by the controller node, e.g. changing health state for the individual and/or setting an alert or instructing the personal device to enable the establishing of a short-range wireless communication link with a mobile communications device associated with the personal device, in accordance with one or more embodiments described herein. The flow 350 may e.g. be as follows:

Step 360: Measuring, by at least one of the plurality of network nodes 300, signal strength values and/or packet loss rates for at least one short-range wireless broadcast signal received from a personal device 200.

Step 370: Receive, by the controller node for a certain personal device, signal strength values and/or packet loss rates for the short-range wireless signals transmitted by the personal device 200 and which are measured and forwarded to the controller node by the at least one of the plurality of network nodes 300.

Step 380: Combine these inputs into a resulting determination, performed by the controller node and/or a backend system receiving the input from the controller node, of whether to take actions in response to the received signal strength values and/or packet loss rates, e.g. to change the health state and/or alert state for the individual wearing the personal device, transmit a message from the controller node (forwarded by the connection node) which contains data instructing the personal device to enable the establishing of a short-range wireless communication link with a mobile communications device associated with the personal device (handover), set an alert or alarm for the personal device and/or individual wearing the personal device, or transmit alert or alarm data associated with the personal device and/or individual wearing the personal device to a local or remote processing arrangement

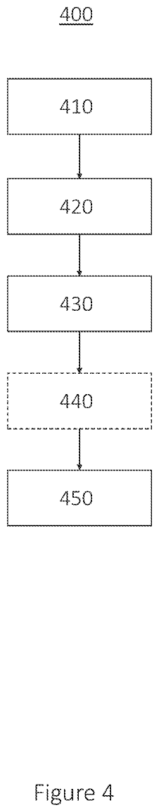

FIG. 4 schematically illustrates a method 400 for monitoring an individual within predetermined facilities 700 in order to determine the current position and/or location for the personal device. The method 400 may include the following steps:

Step 410: transmitting short-range wireless broadcast signals from a personal device 200.

Step 420: measuring, by a plurality of network nodes 300, signal strength values of the short-range wireless broadcast signals transmitted by the personal device 200 and which are received by the plurality of network nodes 300.

Step 430: forwarding the measured signal strength values measured by the respective of the plurality of network nodes 300 to the controller node for the personal device.

Step 450: determining the current position and/or location of the personal device 200 within the facilities 700 based at least on the received signal strength values.

In certain embodiments, the method (400) may comprise:

Step 440: retrieving information from a storage means 320 regarding the layout of the facilities 700 within which the monitoring of the individual takes place.

In these certain embodiments, the step 450 of determining the location of the personal device 200 within the facilities 700 is based at least on the received signal strength values together with the information retrieved from a storage means 320 regarding the layout of the facilities 700 within which the monitoring of the individual takes place.

In the embodiments, the step 450 of determining the current position and/or location for the personal device may be based at least partly on received sensor data, e.g. obtained by a pressure sensor or motion sensor of the personal device, indicates a specific location and/or a specific node to be the new connection node and/or whether the determined location fulfils a predetermined location condition, indicating whether the individual is located in a certain room, such as e.g. the bedroom 630.

The combination of motion information with location information improves the determination of changing connection node for the personal device.

The determining of whether the received sensor data indicates a specific location among a plurality of predetermined location may e.g. be done by a trained machine learning algorithm of the controller node or a locally-installed processing arrangement or a remote processing arrangement such as a backend system communicatively connected to the controller node, since machine learning is an efficient way of evaluating complex signals in situations where large amounts of data is collected and moving patterns of the individuals wearing the personal devices are repetitive and/or predictable.

In embodiments, the distance between the personal device 200 and the at least one network node 300 may e.g. be determined based on the received or obtained signal strength indication (RSSI) of the short-range wireless signal transmitted by the personal device 200 and received by the plurality of network nodes, since the received signal strength will be lower if the distance increases. If the RRSI for more than one network node is determined, the location may be more exactly determined based on e.g. triangulation. Machine learning may also be used for this determination.

In embodiments, the determining 450 of the current position and/or location of the personal device 200 is based also on an indication of the movement of the individual in relation to a previously determined location, based on sensor data from the at least one sensor 220. If the individual is determined to be in a certain location, and the movement required for moving to this location from a previously determined location is more than the movement that has been detected by the at least one movement sensor, it is likely that the determined location is incorrect.

In embodiments, the at least one sensor 220 is an accelerometer. However, other types of motion sensors may also be used.

In embodiments, the personal device 200 is a wristworn device, such as e.g. a wristband. The personal device 200 may however be any type of device that may be worn by an individual, such as e.g. a necklace or a clip.

In embodiments, communication between the personal device 200 and the at least one node 300 takes place using a personal device communication interface 230 and a node communication interface 330. In embodiments, the technology disclosed may include continuously determining and updating an activity state for the monitored individual by continuously receiving sensor data from the personal device, e.g. health sensor data obtained by a medical sensor of the personal device.

The activity state may e.g. categorize the current activity of the individual. Activity states may e.g. be LYING IN BED, LYING ON COUCH, LYING ON FLOOR, SITTING, STANDING, WALKING, RUNNING, RIDING A WHEELCHAIR, WALKING UP/DOWN STAIRS, IN THE BATHROOM. There may also be a specific activity state for when the individual is not wearing the personal device, since no other activity state can then be set. There may be a specific sensor, such as e.g. a contact sensor, a temperature sensor or a heart rate sensor, for determining whether the individual is wearing the personal device. Such a sensor may of course also be used for other purposes.

In embodiments, the technology disclosed may include continuously determining and updating a health state for the monitored individual by continuously receiving sensor data from the personal device worn by the individual, e.g. health sensor data obtained by at least one medical sensor of the personal device.

The determining of the activity state and/or the health state of the individual wearing the personal device may e.g. be done by a trained machine learning algorithm, since machine learning is an efficient way of evaluating complex signals in situations where large amounts of data is collected.

In embodiments, the personal device 200 may also comprise sensors, such as e.g. a motion sensor or pressure sensor. It is known to use a pressure sensor in a personal device to detect a fall based on a change in pressure. However, a pressure sensor may also be involved in the determination, by the controller, to change connection node for the personal device 200 carried by the individual. If the facilities 700 comprise several floors, and the network nodes 300 are arranged in the ceilings or high up on the walls, the RSSI may give erroneous results due to the signals travelling between the floors. In this situation, data obtained by a pressure sensor of the personal device received by the controller node may be used by the controller node to determine the floor on which the individual is located which, in turn, is an input factor to the step of determining, by the controller node for the personal device 200, whether to change connection node for the personal device 200.

In embodiments and in order to improve the determination of whether to change connection node for a personal device 200, the determination of the location and/or whether to change connection node may be improved by involving also signals from a sensor 220 of the personal device 200. The sensor 220 may e.g. be a movement sensor used as a pedometer, so that the signals from the movement sensor 220 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 220, it is likely that the determined location is incorrect. The at least one processing device 310 of a network node (301, 302, 303, 304, 305, 306) may thus be arranged to determine the location of the personal device 200 influencing the decision by the controller node, or processing arrangement, whether to change alert state and/or health state for the personal device 200 based also on an indication of the movement of the individual in relation to a previously determined location, based on sensor data from the at least one sensor 220.

The processing of the sensor data from the at least one sensor 220 may be done by a trained machine learning algorithm. When many individuals are monitored using many personal devices 200, large amounts of sensor data is collected from the sensors 220 in these personal devices 200. Very efficient evaluation of this sensor data may be done using machine learning, in order to determine a suitable machine learning algorithm.

The updating of the activity state and/or health state for the monitored individual may be incremental, so that only differences in relation to the previously determined activity state and/or health state need to be determined.

The determination of the location for the personal device 200 and/or the by the controller node to change connection node for the personal device 200 may e.g. be done by a trained machine learning algorithm. Machine learning is an efficient way of evaluating complex signals in situations where large amounts of data is collected.

When the determining of whether to change alert state and/or health state is based on received sensor data indicates a certain location and whether the determined location fulfils a predetermined location condition, the predetermined location condition may be an absolute condition, such that if the determined location does not fulfil the predetermined location condition, the controller node will, or will not, determine to change alert state and/or health state for the personal device 200. However, there may instead be weighing factors, so that if the received sensor data strongly indicates a specific location, and/or the determined location does not strongly indicate a specific location (that does not fulfil the predetermined location condition), the controller node will, or will not, determine to change alert state and/or health state for the personal device 200.

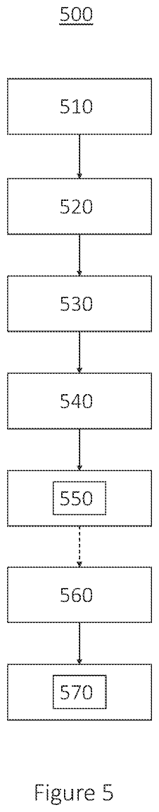

FIG. 5 schematically illustrates embodiments of the technology disclosed including a method (500) for handover of a personal device between a self-organizing network and a mobile communications device and the monitoring of an individual wearing a personal device within and outside predetermined facilities (700) comprising the personal device and a self-organizing network including at least two network nodes, the method (500) comprising:
a. transmitting (510), from the personal device, at least one short-range wireless broadcast signal;
b. receiving (520), by at least one node of the at least two network nodes, the at least one short-range wireless broadcast signal;
c. assigning (530), by the self-organizing network, one node among the at least two network nodes to be controller node for the personal device;
d. assigning (540), by the controller node, a connection node for establishing a short-range wireless communication link between the connection node and the personal device;
e. establishing (550), by the connection node, a short-range wireless communication link with the personal device;
f. enabling (560), by the personal device, the establishing of a short-range wireless communication link between the personal device and a mobile communications device; and
g. establishing (570) a short-range wireless communication link between the personal device and a mobile communications device communicatively coupled to a cellular-type mobile communications network.

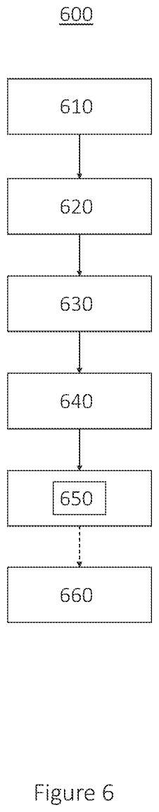

FIG. 6 schematically illustrates embodiments of the technology disclosed including a method (600) for handover of a personal device between a self-organizing network and a mobile communications device and the monitoring of an individual within and outside predetermined facilities (700) having a self-organizing network comprising at least two network nodes and a personal device, the method (600) comprising:
a. establishing (610) a short-range wireless communication link between the personal device and a connection node assigned to the personal device;
b. receiving (620), by the personal device and via the established short-range wireless communication link, at least one short-range wireless signal;
c. measuring (630), by the connection node, packet loss rates and/or signal strengths of the received at least one short-range wireless signal;
d. transmitting (640), from the connection node via the IP-based network connecting the at least two nodes of the node network, measurement data to the controller node, wherein the measurement data indicates the measured packet loss rates and/or signal strengths values;
e. receiving (650), by the controller node assigned to the personal device and via the IP-based network, the transmitted measurement data indicating the measured packet loss rates and/or signal strengths values; and
f. determining (660), by the controller node, to transmit a message to the personal device containing data instructing the personal device to enable the establishment of a short-range wireless communication link with a mobile communications device, wherein the determining to transmit the message is in response to and/or based on the measurement data received from the connection node is indicating that the established short-range wireless communication link is poor.

In aspects, the system is configured so that the new connection node may be a different node from the current controller node for the personal device but may also be the controller node for the personal device.

In embodiments of the technology disclosed, the system is configured so that the connection node assigned to a personal device is never the assigned controller node for the personal device.

In aspects, the technology disclosed relates to a method and system for monitoring an individual within predetermined facilities (700):
receiving, by the sole connection node assigned to the personal device, a message comprising sensor data obtained by at least one sensor (220) arranged in the personal device (200);
forwarding, by the connection node and to the controller node assigned to the personal device, a message comprising the sensor data or data reflecting the sensor data;
determining, by the controller node whether the received sensor data indicates certain event data, a specific health state and/or a specific alert state among a plurality of predetermined alert states, where the alert states may comprising at least a FALL alert state, to be used if a probable fall has been detected for the monitored individual, and an OUT OF BED alert state, to be used if it has been detected that the monitored individual is probably getting out of bed;
receiving, by a plurality of nodes within short-range wireless communication range with the personal device, short-range wireless broadcast signals from the personal device;
measuring, by each of the plurality of nodes, signal strengths values of the received short-range wireless broadcast signals;
forwarding, by each of the plurality of nodes and to the controller node assigned to the personal device, a message containing the signal strengths values of the received short-range wireless broadcast signals;
determining, by the controller node, the location of the personal device (200) within the facilities (700) based at least on the received signal strengths values of the short-range wireless broadcast signals together with information retrieved from a storage means (320) regarding the layout of the facilities (700) within which the monitoring of the individual takes place; and
setting an alert and/or change a health state based at least on whether the received sensor data indicates a specific event, health state and/or an alert state and whether the determined location fulfils a predetermined location condition, indicating whether the individual is located in a certain room, such as e.g. the bedroom (630).

According to embodiments of the various methods mentioned above, the determining of the location of the personal device (200) is based also on an indication of the movement of the individual in relation to a previously determined location, based on sensor data from the at least one sensor (220), e.g. a motion sensor and/or a pressure sensor.

According to embodiments of the technology disclosed and mentioned above, the method further comprising continuously determining and updating an activity state and/or a heath state for the monitored individual.

According to embodiments of the technology disclosed and mentioned above, the method further the change of health state and/or setting of the alert is based also on at least one previously determined health state or activity state. In the example of setting an alert state, the setting of an OUT OF BED alert state may also depend on whether the previous activity state was a LYING IN BED alert state, or so that the setting of a FALL alert state also depends on that the previous activity state was not any type of LYING activity state.

According to embodiments of the technology disclosed and mentioned above, the changed health state and/or setting of the alert is based also on the time period for which the individual has had a specific activity state, such as e.g. for how long the individual has been in the activity state STANDING.

According to embodiments of the technology disclosed and mentioned above, determining of the changed health state and/or activity state is done by a trained machine learning algorithm.

According to embodiments of the technology disclosed and mentioned above, the method further comprises sending an alarm signal if a predetermined alert or health state is set.

In embodiments, the communication between the personal device (200) and the at least one network node (300) takes place using a personal device communication interface (230) and a network node communication interface (330).

In aspects, the technology disclosed relates to a system (100) for monitoring an individual within predetermined facilities (700), the system (100) comprising:
a personal device (200) to be worn by the monitored individual, the personal device (200) comprising at least one sensor or input means (220);
at least one network node (300), with which the personal device (200) is arranged to communicate via an established short-range wireless connection;
a storage means (320); and
at least one processing device (210, 310, 160), arranged to:
   receive sensor data, event data and/or input data from the at least one sensor and/or input means (220) in the personal device (200);
   determine whether the received sensor data, event data and/or input data indicates a specific health state and/or an alert state among a plurality of health states and/or predetermined alert states; and;
   set a health state and/or an alert based at least on whether the received sensor data, event data and/or input data indicates a health state and/or an alert state.

In embodiments, the system (100) and controller node for the personal device (200) are configured to set a health state and/or an alert based at least on whether a determined location fulfils a predetermined location condition, indicating whether the individual is located in a certain room, such as e.g. the bedroom (630).

In embodiments, the system (100) and controller node for the personal device (200) are configured to determine the distance between the personal device (200) and the at least one network node (300), e.g. based on signal strength values measured by at least one of the network nodes.

In embodiments, the system (100) and controller node for the personal device (200) are configured to determine the location of the personal device (200) within the facilities (700) based at least on the determined distance together with information retrieved from the storage means (320) regarding the layout of the facilities (700) within which the monitoring of the individual takes place.

In embodiments, the system (100) and the at least one processing device (210, 310, 160) are arranged to continuously determine and update a health state and/or an activity state for the monitored individual based on received sensor data is indicating a specific event, health state and/or an alert state.

In embodiments, the personal device (200) comprises at least one motion sensor (220). In certain embodiment the at least one motion sensor (220) is an accelerometer.

In embodiments, the personal device (200) comprises at least one medical sensor (220), or health sensor, where the medical sensor (220) is adapted to obtain health sensor data to be used for determining the current health status or health state of the individual wearing the personal device.

In embodiments, the personal device (200) is a wristworn device, such as e.g. a wristband, clip or necklace.

An accelerometer, is a device that measures proper acceleration. Proper acceleration, being the acceleration (or rate of change of velocity) of a body in its own instantaneous rest frame, is not the same as coordinate acceleration, being the acceleration in a fixed coordinate system.

The input from the movement sensor based on sensor data indicating an acceleration beyond a certain threshold value and/or a certain changed movement pattern for the personal device may trigger the short-range wireless transmission of event data from the personal device.

In aspects, the technology disclosed proposes a system and node network for dynamically assigning roles to the nodes of the network on two levels, a control level and connect level. Each of the personal device may then be assigned only one network node, on the control level, to be controller node for the personal device and only one network node, on the connect level, to be the connection node for the personal device. The assigned controller is then responsible for dynamically assigning only one connection node for the personal device at a time. The controller node assigned to a personal device may be assigned according to a common decision model known to and used by the at least two network nodes and following the sharing or distribution of data associated with the personal device. In embodiments, the data shared between the nodes may then be used as input values to the common decision model for dynamically changing controller node for the personal device.

According to aspects of the technology disclosed, there is only one connection node for establishing a short-range wireless connection with the personal device and the system is configured so that the controller node for the personal device has the sole responsibility for changing connection node for the personal devices. This provides for a fast response yet robust and scalable solution for monitoring personal devices, e.g. wristbands, necklaces or clips carried or worn by individuals moving between locations or rooms of a facility such as patients in a nursing home. The established short-range wireless connection is then the only connection between the network and the personal device. According to this embodiment, the personal device may then still transmit short-range wireless broadcast signals to provide the other network nodes within short-range wireless communication range with status data, obtained sensor data, event data and/or input data associated with the personal device.

According to aspects of the technology disclosed, the data transmitted from the personal devices to the node network and between the personal devices and the network nodes may then contain sensor data or event data obtained and transmitted by the personal devices and then received by at least one network node within short-range wireless communication range. The transmission of data between the at last two network nodes may include at least one of the transmission of data associated with a certain personal device which is transmitted or forwarded from one of the nodes to the assigned controller node for the personal device, transmission of data which is shared between the nodes prior to assigning a node to be the controller node for a personal device and the transmission of data from the controller node to inform and/or instruct a (new) connection node which is assigned, e.g. dynamically assigned, by the controller node to be the connection node for a personal device and which is assigned to establish a short-range wireless connection with the personal device.

The technology disclosed provides a more scalable system, e.g. monitoring system, comprising a node network that does not rely on the presence of other personal devices within short-range communication range for making decisions on behalf of a personal device and, since the personal devices connected to the node network are not sharing their short-range wireless communication interface to the node network with short-range wireless connections to other personal devices, is less dependent on the currently available short-range wireless bandwidth. The technology disclosed comprising a node network where the nodes are transmitting obtained data associated with a certain personal device to the controller node responsible for the personal is therefore less dependent on the current number of other short-range wireless connections to the communication interface of the personal devices.

According to aspects, the controller node having the sole responsibility for a certain personal device is responsible for assigning and dynamically changing which of the at least two network nodes is the connection node for the personal device. The controller node may then decide to change connection node for a personal device the controller node is responsible for at least partly based on data received from the other nodes, e.g. measured signal strengths of short-range broadcast signals or messages transmitted by the personal device and which are received by any of the other nodes which are within short-range communication range with the personal device. The controller node may thereby see to that the personal device always has the best possible or available short-range wireless connection, e.g. Bluetooth connection, to the network of nodes which in turn may be communicatively coupled to a backend system. This provides for a scalable system which responds quickly to events, obtained sensor data and the movements of the individuals in that data is shared between the nodes, yet is robust in that each of the personal devices is continuously and always provided with a reliable short-range wireless connection to the network.

In embodiments, the established short-range wireless communication link between the connection node and the personal device is a bidirectional short-range wireless communication link.

In embodiments, the established short-range wireless communication link between the connection node and the mobile communications device is a bidirectional short-range wireless communication link.

In embodiments, each of the plurality of personal devices is configured to transmit data indicating the same obtained sensor data and/or event data both via their respective established short-range wireless connection with their respectively assigned connection node and via short-range wireless broadcast signals. The same sensor data and/or event data may then be data indicating at least one of the activation of an input means of the personal device, data indicating a trigger event and sensor data indicating a health state and/or alert state for the personal device and/or the individual wearing the personal device.

In embodiments, the controller node for the respective personal device is configured to determine a specific alert state and/or a health state for the individual wearing the personal device at least partly based on received sensor data and/or event data associated with the personal device.

In certain embodiments, the controller node for the respective personal device is configured to set or change a health state for the individual wearing a personal device at least partly based on received sensor data obtained by and/or originating from at least one medical sensor of the personal device.

In embodiments, the assignment of a controller node may use a common decision model which is known to the nodes of the node network where the decision model may, or may not, use input or parameter data values obtained from the personal devices and/or other nodes of the network to determine which of the at least two nodes is going to be the controller node. The input or parameter data values may change the output from the use of the common decision model and the determining of which of the network nodes is going to be the controller node for a personal device. In some embodiments, the controller node assigned to a certain personal device may be dynamically changed at least partly based on obtained input or parameter data values.

In embodiments, the assignment, by the controller node, of a connection node may be at least partly based on signal strengths of short-range wireless broadcast signals which are measured by a plurality of network nodes within short-range communication range with the personal device transmitting the short-range wireless broadcast signals. The assignment of connection node by the controller node may further be based on or use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

In embodiments, the technology disclosed relates to a personal device, a system comprising at least two network nodes and methods in a system comprising a Self-Organizing Network (SON) and a plurality of personal devices, e.g. a plurality of wristbands worn by individuals such as patients.

In aspects and certain embodiments, the technology disclosed relates to a system including a network comprising at least two network nodes configured to transmit data to the other nodes via the network and a plurality of personal devices with short-range wireless communication capabilities for communicating with the at least two network nodes. The plurality of personal devices are each assigned only one controller node among the at least two network nodes, and the respective controller node is responsible for assigning a connection node among the at least two network nodes to establish a short-range wireless communication connection with a personal device the controller node is responsible for.

In embodiments, the system, e.g. monitoring system, is configured so that each of the plurality of personal devices is assigned only one controller node which has the sole responsibility for the personal device.

In embodiments, the system is configured so that each or at least one of the at least two network nodes may be the assigned sole controller node for a plurality of personal devices.

In embodiments, the system including the at least two network nodes is configured to use a common decision model which is known to each of the at least two nodes for determining which of the at least two nodes is going to be the controller node assigned to the respective personal device.

In embodiments, the common decision model use input values or parameters which are continuously and dynamically changed at least partly based on data distributed and shared between the network nodes, thereby allowing for the determining of which of the nodes among the at least two nodes is going to be the controller node for the personal device to be dynamically changed at least partly on data distributed and shared between the network nodes. The network for distributing and sharing data between the network nodes is typically an IP-based network providing for improved scalability, e.g. a WiFi or Ethernet.

In embodiments, the system is configured so that each assigned network node to be connection node for a personal device only has one established short-range wireless communication connection with the node network at a time.

In embodiments, the plurality of personal devices are each configured to be transmitting short-range wireless broadcast signals while having an established short-range wireless communication connection with their respective connection node.

In embodiments, the only one controller node assigned to the respective personal device is configured to assign and dynamically change which of the at least two network nodes is currently the only one connection node for the personal device at least partly based on at least one of contents and received signal strengths of at least one short-range wireless broadcast signal transmitted from the personal device and which is received by a plurality of other nodes than the controller node.

In embodiments, the only one controller node assigned to the respective personal device is configured to receive data from a plurality of other nodes than the controller node via an IP-based network connecting the at least two network nodes, and wherein the data received via the IP-based network include data indicating the measured signal strength values of short-range wireless broadcast signals transmitted by the respective personal device and which are received and measured by the plurality of other nodes.

In certain embodiments, the technology disclosed relates to methods and a system for monitoring an individual within predetermined facilities by identifying an event associated with a personal device carried or worn by the individual, e.g. with the purpose of determining whether there is a need for setting an alert. The system may comprise at least two network nodes and a plurality of personal devices each carried or worn by the respective monitored individual, where each of the plurality of personal devices comprises at least one sensor and has short-range communication capability, e.g. Bluetooth communication capability, with which the respective personal device is arranged to communicate with the network nodes. The personal device may also comprise a storage means and at least one processing device.

In embodiments, the personal device may be a wristband and the at least one sensor of the personal device may comprise at least one medical sensor, or health sensor, adapted for obtaining sensor data for determining the medical and/or physical condition of the wearer of the wristband, e.g. a medical sensor configured to sense a characteristic of the user and generate a sensor output signal.

In embodiments, the personal device may be a wristband and the at least one sensor of the personal device may comprise at least one health sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a body temperature sensor for obtaining sensor data for determining the body temperature of the wearer, or may include a blood pressure sensor, a heart rate sensor, a skin galvanometric level sensor etc.

In embodiments, the at least one processing device of the personal device may be arranged to receive sensor data from the at least one sensor in the personal device and determine whether the received sensor data indicates an alert state, e.g. a specific alert state among a plurality of predetermined alert states.

In certain embodiments, the alert states may comprise at least a FALL alert state to be used if a probable fall has been detected for the monitored individual and an OUT OF BED alert state to be used if it has been detected that the monitored individual is probably getting out of bed.

According to certain aspects of the technology disclosed, personal devices communicate with nodes in the node network using Bluetooth, e.g. Bluetooth broadcasts or via an established Bluetooth connection, whereas the communication between the network nodes takes place using the IP addresses of the nodes (e.g. via Wi-Fi). The network of nodes may then be self-organizing in that the nodes of the network continuously and/or automatically assigns roles to the individual nodes, where the assignment of a role is preceded by, and at least partly based on, data distributed and/or shared between the network nodes. The distributed and/or shared data is obtained by the other network nodes and is associated with the personal device to which the assignment of a role concerns.

The personal devices may broadcast their own universal unique identifiers (UUIDs) and seek to establish connections with Bluetooth devices within its Bluetooth communication range. The network nodes may be configured to store the UUIDs of all personal devices detected in their memories. The connection node assigned, by the controller node, to a certain personal device may then establish a Bluetooth connection with the personal device by transmitting a connection request via Bluetooth to the personal device where the connection request may include the unique identifier (UUID) of the personal device.

According to aspects, the network nodes may be self-organizing in that the assignment of the one controller node having the sole responsibility for a specific personal device is a distributed decision which is preceded by at least one of distributing and sharing data between the network nodes using the IP-based network and by using a predetermined and common decision model with dynamic input values/parameters which are changed with the data distributed and shared between the network nodes.

According to embodiments, the network nodes may be self-organizing in that the decision to change controller node for a specific personal device is a distributed decision which is preceded by at least one of distributing and sharing of data between the network nodes using the IP-based network and by using a predetermined and common decision model with dynamic input values/parameters which are changed with the data distributed and shared between the network nodes.

According to aspects of the technology disclosed, personal devices transmits data to the nodes in the node network using Bluetooth, e.g. via Bluetooth broadcasts and an established Bluetooth connection with one of the nodes, and the transmission of data, e.g. data associated with the individual personal device, takes place using their IP addresses, e.g. data received from a certain personal device or associated with a certain personal device may be shared between network nodes via the IP-based network connecting the network nodes.

In embodiments, the technology disclosed relates to a method for a system including a network, e.g. a Self-Organizing Network (SON), comprising at least two network nodes configured to share data with the other nodes via an IP-based network, e.g. an Ethernet or WiFi network, and a plurality of personal devices with Bluetooth communication capabilities, the method comprising:
a. assigning only one controller node among the at least two network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node; and
e. forwarding, by the connection node and via the IP-based network, the data received from the personal device to the assigned controller node for the personal device.

In embodiments, the technology disclosed relates to a method for assigning a connection node for a personal device in a Self-Organizing Network (SON) comprising at least two network nodes configured to share data with the other nodes via an IP-based network, e.g. an Ethernet or WiFi network, and a plurality of personal devices with Bluetooth communication capabilities, the method comprising:
a. assigning one of the at least two network nodes to be the controller node with sole responsibility for a personal device;
b. transmitting, by the personal device not having an established connection with any of the at least two network nodes, Bluetooth broadcast messages/signals;
c. receiving, by a plurality of the at least two network nodes within Bluetooth communication range, at least one Bluetooth broadcast message/signal from the personal device, where the at least one Bluetooth broadcast message/signal contains unique identification data for the personal device;
d. forwarding, from the plurality of nodes to the assigned controller node via the IP-based network, position data for the personal device and/or measured signal strength values for the personal device, wherein the position data and/or measured signal strength values are obtained through the plurality of nodes receiving the at least one Bluetooth broadcast message/signal;
e. determining, by the node assigned to be the controller node with sole responsibility for the personal device, to assign only one node among the at least two network nodes to be the sole connection node for the personal device, wherein the determining by the controller node of which node will be the connection node for the personal device is at least partly based the position data and/or measured signal strength values obtained through the plurality of nodes receiving the at least one Bluetooth broadcast message/signal; and
f. establishing, by the node determined by the controller node to be connection node for the personal device, a Bluetooth connection with the personal device.

In embodiments, the technology disclosed relates to a method for changing the connection node for establishing a Bluetooth connection with a certain personal device in a Self-Organizing Network (SON) comprising at least two network nodes configured to share data with the other nodes via an IP-based network, e.g. an Ethernet or WiFi network, and a plurality of personal devices with Bluetooth communication capabilities, the method comprising:
a. assigning only one controller node among the at least two network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node;
e. forwarding, by the connection node and via the IP-based network, the data received from the personal device (via the established Bluetooth connection) to the assigned controller node for the personal device;
f. transmitting, by the personal device, Bluetooth broadcast messages/signals, where the Bluetooth broadcast messages/signals are received by any of the at least two node which is within Bluetooth communication range; and
g. determining, by the controller node the personal device, a position and/or location for the personal device, where the determining of the position and/or location is at least partly based on measured signal strength values for Bluetooth broadcast messages/signals received by other nodes than the controller node and the current connection node.

In aspects, the technology disclosed relates to system comprising at least two network nodes and a plurality of personal devices, where each of the personal devices is assigned/having only one controller node and only one connection node at a time. The controller node for a personal device may then be assigned by sharing data between the nodes of the network and using a predetermined decision model, e.g. a distributed consensus model which is known to all of the at least two network nodes. In embodiments, the predetermined decision model may have dynamic input values/parameters, where the input values/parameters may include, for example, data related to load balancing between different parts of the network and an assessment of the current traffic load distribution between the at least two nodes of the IP-based network.

In aspects, the technology disclosed relates to a method in a system, e.g. a monitoring system, including a network comprising at least two network nodes configured to share data with the other nodes via an IP-based network and a plurality of personal devices with short-range wireless communication capabilities, the method comprising:
a. assigning only one controller node among the at least two network nodes to have the sole responsibility for a personal device;
b. assigning, by the controller node, only one connection node for the personal device, wherein the controller node is sole responsible for assigning the only one connection node to the personal device;
c. establishing, by the assigned only one connection node, a short-range wireless communication connection between the only one connection node and the personal device, wherein the short-range wireless communication connection is the only currently established connection from the network to the personal device;
d. transmitting, from the personal device, data via the established short-range wireless communication connection to the only one connection node; and
e. transmitting, from the personal device, short-range wireless broadcast signals, wherein the short-range wireless broadcast signals are transmitted while the personal device has the established Bluetooth connection with the only one connection node.

In embodiments, the method according to the technology disclosed is comprising:
a. determining, by the only one controller node, the current position of the personal device based on the received data indicating the Bluetooth broadcast signal strengths values measured by a plurality of nodes.

In embodiments, the method according to the technology disclosed is comprising:
a. determining, by the personal device, that a trigger event has occurred, wherein the
b. wherein each of the plurality of personal devices is configured to transmit Bluetooth broadcast signals including alarm data in response to a trigger event determined by the respective personal device to have occurred, and wherein the alarm data is adapted to indicate the occurrence of the trigger event to any node receiving the Bluetooth broadcast signal and is transmitted by the respective personal device while having an established Bluetooth connection with their respectively assigned only one connection node.

In embodiments, the system comprises at least one mobile communication device and a backend system and network configured to receive alarm data indicating the occurred trigger event from the only one controller node responsible for the personal device which determined that the trigger event occurred, and wherein the backend system and network is further configured to transmit alarm data to the at least one mobile communication device in response to receiving the alarm data from the only one controller node.

In embodiments, the backend system and network is further configured to transmit the alarm data to at least one mobile device, e.g. at least one mobile device of a staff member of the facility, via a network separate from the IP-based network used by the at least two network nodes to exchange data.

In embodiments, the trigger event is the activation of an input means on the respective personal device. The personal device may be a wristband and the trigger event may be the action of pressing a button on the wristband or activating a voice activation means of the personal device.

In embodiments, the only one controller node assigned to the respective personal device is configured to receive data via the network, e.g. an IP-based network, from a plurality of other nodes than the controller node. The data received via the IP-based network may then include data indicating the measured signal strength values of Bluetooth broadcast signals transmitted by the respective personal device and which are received and measured by the respective node of the plurality of other nodes.

In embodiments, the only one controller node assigned to the respective personal device is further configured to determine the current position and/or location of the personal device it is responsible for based on the data indicating the measured signal strength values of Bluetooth broadcast signals transmitted by the respective personal device and which are received and measured by the plurality of other nodes.

In embodiments, the system, e.g. monitoring system, further comprises a local or remote processing arrangement such as a backend system and network configured to receive data indicating measured Bluetooth broadcast signal strength values for a personal device from the only one controller node responsible for the personal device. The backend system and network may then be configured to determine the current position for the personal device based on the received Bluetooth broadcast signal strength values for the personal device and return the determined position to the only one controller node responsible for the personal device.

In embodiments, each of the plurality of personal devices is configured to transmit Bluetooth broadcast signals including alarm data in response to a trigger event determined by the respective personal device to have occurred, wherein the alarm data is adapted to indicate the occurrence of the trigger event to any node within short-range wireless communication range receiving the Bluetooth broadcast signal and is transmitted by the respective personal device while having an established Bluetooth connection with their respectively assigned only one connection node.

In embodiments, each of the plurality of personal devices is further configured to, in addition, transmit data including redundant alarm data in response to the determined occurrence of the trigger event via the established Bluetooth connection with their respectively assigned only one connection node. The respectively assigned only one connection node may then be configured to, in response to receiving the alarm data via the established Bluetooth connection, at least one of transmit and forward data including alarm data via the network, e.g. IP-based network, to the respectively assigned only one controller node, where the alarm data is adapted to indicate to the only one controller node that the trigger event has occurred.

In embodiments, the system comprises at least one mobile communication device and a backend system and network configured to receive alarm data indicating the occurred trigger event from the only one controller node responsible for the personal device which determined that the trigger event occurred. The backend system may then be configured to transmit alarm data to the at least one mobile communication device of an individual such as a staff member of the facility in response to receiving the alarm data from the only one controller node.

In embodiments, the backend system and network is further configured to transmit the alarm data to the at least one mobile communication device via a network separate from the network, e.g. IP-based network, used by the at least two network nodes to exchange data.

1: A method in a system including a network comprising at least two network nodes and at least one personal device with short-range wireless communication capabilities, the method comprising:
transmitting, from a personal device, at least one short-range wireless broadcast signal;
receiving, by at least one node of the at least two network nodes, said at least one short-range wireless broadcast signal;
assigning one node among the at least two network nodes to be controller node for said personal device;
assigning a connection node for establishing a short-range wireless communication link between said connection node and said personal device;
establishing, by said assigned connection node, a short-range wireless communication link with said personal device;
transmitting, from said personal device and via said established short-range wireless communication link, a message to said connection node;
receiving, by said connection node, said message, wherein said message contains data including at least one of status data, sensor data and event data associated with said personal device;
transmitting, from the assigned connection node to the controller node for said personal device, data including said at least one of status data, sensor data and event data associated with said personal device;
enabling, by said personal device, the establishing of a short-range wireless communication link between said personal device and a mobile communications device; and
establishing a short-range wireless communication link between said personal device and a mobile communications device communicatively coupled to a cellular-type mobile communications network.

2: The method of claim 1, wherein said step of enabling, by said personal device, the establishing of a short-range wireless communication link with a mobile communications device is in response to and triggered by a timer duration period of a timer of said personal device is exceeded without said personal device receiving any messages over said short-range wireless communication link.

3: The method of claim 1, wherein said step of enabling, by said personal device, the establishing of a short-range wireless communication link with a mobile communications device is followed by the step of:
receiving, by said personal device and from a mobile communications device, a connection request in the form of a broadcast signal for establishing a short-range wireless communication link with the mobile communications device.

4: The method of claim 1, wherein said step of enabling, by said personal device, the establishing of a short-range wireless communication link with a mobile communications device is in response to and triggered by a timer duration period of a timer of at least one of said assigned connection node, said assigned controller node and a locally-installed or remote processing arrangement communicatively coupled to said controller node is exceeded without said assigned connection node receiving any messages over said short-range wireless communication link.

5: The method of claim 4, wherein said step of enabling, by said personal device, of a short-range wireless communication link with a mobile communications device is in response to and triggered by:
receiving, by said personal device and via said established short-range wireless communication link with said connection node assigned to said personal device, a message containing data adapted to instruct said personal device to enable a short-range wireless communication link with a mobile communications device.

6: The method of claim 5, wherein said message and/or instruction data is transmitted from at least one of said controller node assigned to said personal device and a locally-installed or remote processing arrangement which is communicatively coupled to said controller node assigned to said personal device.

7: The method of claim 1, wherein said step of establishing a short-range wireless communication link between said personal device and the mobile communications device is preceded by and in response to the following steps:
transmitting, from said personal device, at least one short-range wireless broadcast signal containing a flag indicating that said personal device allows the establishing of a short-range wireless communication link with a mobile communications device; and
receiving, by a mobile communications device connected to a cellular mobile communications network, said at least one short-range wireless broadcast signal containing said flag.

8: The method of claim 7, wherein said mobile communications device transmits a connection request to said personal device in response to receiving said at least one short-range wireless broadcast signal containing said flag indicating that said personal device allows the establishing of a short-range wireless communication link with the mobile communications device.

9: The method of claim 7, wherein said flag of said at least one short-range wireless broadcast signal is adapted to indicate only to a mobile communications device which is associated with, or known to, the personal device that said personal device allows said establishing of a short-range wireless communication link, and wherein said flag does not indicate to other mobile communications devices which are not associated with said personal device that they can establish a short-range wireless communication link with said personal device.

10: The method of claim 7, wherein said step of transmitting at least one short-range wireless broadcast signal containing a flag is preceded by the step of:
determining, by said personal device, that said short-range wireless communication link is disconnected based on a preset timer duration period is exceeded without receiving any messages over said short-range wireless communication link.

11: The method of claim 10, wherein said at least one short-range wireless broadcast signal containing a flag is transmitted in response to said step of determining that said short-range wireless communication link is disconnected based on a preset timer duration period is exceeded without receiving any messages over said short-range wireless communication link.

12: The method of claim 1, further comprising the following step after establishing of said short-range wireless communication link between said personal device and the mobile communications device:
enabling, by said personal device, a second simultaneous short-range wireless communication link.

13: The method of claim 7, wherein said second short-range wireless communication link is established in response to and following the further step of:
determining, by said controller node, that the link condition of said short-range wireless communication link is poor based on at least one of low signal strength values and high packet loss rates over said short-range wireless communication link obtained by said connection node.

14: The method of claim 13, wherein said at least one short-range wireless broadcast signal containing a flag is transmitted in response to said step of determining that the link condition of said short-range wireless communication link is poor based on at least one of low signal strength values and high packet loss rates over said short-range wireless communication link obtained by said connection node.

15: The method of claim 13, wherein said second short-range wireless communication link with the mobile communications device is established while said short-range wireless communication link with the connection node is still active and/or has not been actively disconnected.

16: The method of claim 13, further comprising the following step preceding the step of transmitting at least one short-range wireless broadcast signal containing a flag:
transmitting, from said controller node via said connection node to said personal device, a message containing data instructing said personal device to enable the establishing of a simultaneous short-range wireless communication link with a mobile communications device.

17: The method of claim 1, wherein said personal device has an established short-range wireless communication link with the mobile communications device, further comprising the step of:
receiving, by one of the at least two network nodes of said network, at least one short-range wireless broadcast signal from said personal device.

18: The method of claim 17, further comprising:
determining, by the network, that the signal strength values of the at least one short-range wireless broadcast signal from said personal device is above a pre-determined signal strength threshold value or below a pre-determined packet loss threshold value;
establishing a new short-range wireless communication link between said personal device and said network by assigning a controller node and a connection node to said personal device.

19: The method of claim 18, further comprising:
determining, by said controller node, that the link condition of said short-range wireless communication link between said personal device and said connection node is above a pre-determined threshold value, again based on at least one of signal strength values and packet loss rates over said short-range wireless communication link obtained by said connection node; and
re connecting said personal device to said network, wherein said re-connecting step is performed in response to and is preceded by said step of determining that the link condition is above a pre-determined threshold value.

20: The method of claim 19, further comprising disconnecting said short-range wireless communication link between said personal device and said mobile communications device.

21: The method of claim 20, wherein said short-range wireless communication link is disconnected in response to and following said establishing of a new short-range wireless communication link between said personal device and said network.

22: A personal device with short-range wireless communication capabilities for use in a system including a local network comprising at least two network nodes, said system is configured to assign a connection node among said at least two network nodes to said personal device, wherein the connection node is responsible for establishing a short-range wireless communication link with their respective personal device, and each of said plurality of personal devices is configured to transmit short-range wireless broadcast signals while their respective established short-range wireless connection with their respectively assigned connection node is active,
wherein said personal device is configured to:
transmit at least one short-range wireless broadcast signal;
establish a first short-range wireless communication link with a connection node of the network, and
transmit at least one short-range wireless broadcast signal containing a flag indicating that the personal device allows the establishing of a short-range wireless communication link with a mobile communications device connected to a cellular mobile communications network.

23: The personal device of claim 21, further comprising a timer and configured to:
determine that said short-range wireless communication link with a connection node of the network is disconnected based on a preset timer duration period of the timer is exceeded without receiving any messages over said short-range wireless communication link.

24: The personal device of claim 23, wherein said personal device is further configured to transmit said at least one short-range wireless broadcast signal containing a flag in response to said step of determining that said short-range wireless communication link is disconnected based on a preset timer duration period is exceeded without receiving any messages over said short-range wireless communication link.

25: The personal device of claim 22, wherein said personal device is configured to establish the short-range wireless communication link with the mobile communications device while said short-range wireless communication link with the connection node has not been actively disconnected by said personal device.

26: The personal device of claim 22, wherein said personal device is further configured to:
receive, from the assigned controller node via the established short-range wireless communication link with said connection node assigned to said personal device, a message containing data instructing said personal device to allow for the establishing of a short-range wireless communication link with a mobile communications device connected to a cellular mobile communications network; and
establish a second simultaneous short-range wireless communication link with a mobile communications device connected to a cellular mobile communications network.

27: A system including a local network comprising at least two network nodes and at least one personal device, said system is configured to assign a connection node among said at least two network nodes to each of the at least one personal device, wherein the assigned connection node is responsible for establishing a short-range wireless communication link with their respective personal device, and each of the at least one personal device is configured to transmit short-range wireless broadcast signals while having an active short-range wireless connection with a connection node assigned to the personal device, said system is further configured to:
establish a short-range wireless communication link between a personal device and a connection node assigned to said personal device;
obtain, by the connection node assigned to said personal device, signal strength values and/or packet loss rates over said short-range wireless communication link;
determine, by the controller node assigned to said personal device, that a link condition of said short-range wireless communication link is poor based on at least one of the signal strength values and/or high packet loss rates obtained by the connection node; and, in response to said determining that the link condition is poor:
transmit, from said controller node assigned to said personal device via said established short-range wireless communication link between said connection node and said personal device, a message containing data instructing said personal device to allow for the establishing of a short-range wireless communication link with a mobile communications device connected to a cellular mobile communications network, and
establish a second simultaneous short-range wireless communication link between said personal device and a mobile communications device connected to a cellular mobile communications network.

28: The system of claim 27, wherein said assigned controller node is a different node from the assigned connection node, and said system is further configured to perform the following step before determining the link condition:
transmit, or forward, by the assigned connection node, the obtained signal strength values and/or packet loss rates to the assigned controller node.
